(19) United States
(12) Patent Application Publication
(10) Pub. No.: US 2026/0018284 A1
(43) Pub. Date: Jan. 15, 2026

(54) SYSTEM FOR REMOTE TREATMENT UTILIZING PRIVACY CONTROLS

(71) Applicant: ROM TECHNOLOGIES, INC., Brookfield, CT (US)

(72) Inventors: DANIEL POSNACK, Fort Lauderdale, FL (US); PETER ARN, Roxbury, CT (US); S. ADAM HACKING, Nashua, NH (US); MICHEAL MUELLER, Oil City, PA (US); JOSEPH GUANERI, Merrick, NY (US); JONATHAN GREENE, Denver, CO (US)

(73) Assignee: ROM TECHNOLOGIES, INC., Brookfield, CT (US)

(21) Appl. No.: 19/335,805

(22) Filed: Sep. 22, 2025

Related U.S. Application Data
(63) Continuation of application No. 18/165,756, filed on Feb. 7, 2023, now Pat. No. 12,424,319.
Application No. 18/165,756 is a division of application No. 17/075,049, filed on Oct. 20, 2020.
(60) Provisional application No. 62/931,278, filed on Nov. 6, 2019.

(51) Int. Cl. G16H 40/67; G06F 21/62; G16H 15/00; G16H 20/30; G16H 80/00

(52) U.S. Cl. CPC G16H 40/67; G06F 21/6254; G16H 15/00; G16H 20/30; G16H 80/00

(57) ABSTRACT

A method for displaying clinician user interface generated by a computer is disclosed. The method may include, displaying clinician user interface generated by a computer. The method may also include a profile display presenting information regarding a treatment history of a patient. The method may also include a protocol management display presenting a treatment plan, with the treatment plan comprising a plurality of treatment protocols. The method may also include a plan modification control configured to modify the plurality of treatment protocols of the treatment plan. The method may include a login interface configured to enable a person to access the clinician user interface by providing a credential associated with one of a plurality of user accounts. The method may include wherein each of the plurality of user accounts has a corresponding set of permissions controlling access to patient data on the clinician user interface.

### CROSS-REFERENCES TO RELATED APPLICATIONS

This application is a continuation of and claims priority to and the benefit of patent application Ser. No. 18/165,756 filed Feb. 7, 2023, titled “System for Remote Treatment Utilizing Privacy Controls, which is a divisional of and claims priority to and the benefit of patent application Ser. No. 17/075,049 filed Oct. 20, 2020, titled “System for Remote Treatment Utilizing Privacy Controls,” which claims priority to and the benefit of U.S. Provisional Application Patent Ser. No. 62/931,278 filed Nov. 6, 2019, titled “System for Remote Treatment Utilizing Privacy Controls,” the entire disclosures of which are hereby incorporated by reference for all purposes.

### BACKGROUND

Patients may use treatment apparatuses for any suitable purpose, such as rehabilitation of a body part, pre-habilitation of a body part, strengthening a body part, exercising a body part, and the like.

### SUMMARY

A method for a clinician user interface generated by a computer is provided. The clinician user interface comprises a protocol management display presenting a treatment plan, with the treatment plan comprising a plurality of treatment protocols. The clinician user interface also comprises a plan modification control configured to modify the plurality of treatment protocols of the treatment plan, and a login interface configured to enable a person to access the clinician user interface by providing a credential associated with one of a plurality of user accounts. Each of the plurality of user accounts has a corresponding set of permissions controlling access to patient data on the clinician user interface.

A computer-implemented system for a clinician user interface generated by a computer is also provided. The clinician user interface comprises a protocol management display presenting a treatment plan, with the treatment plan comprising a plurality of treatment protocols. The clinician user interface also comprises a plan modification control configured to modify the plurality of treatment protocols of the treatment plan, and a login interface configured to enable a person to access the clinician user interface by providing a credential associated with one of a plurality of user accounts. Each of the plurality of user accounts has a corresponding set of permissions controlling access to patient data on the clinician user interface.

A clinician user interface generated by a computer is also provided. The clinician user interface comprises a protocol management display presenting a treatment plan, with the treatment plan comprising a plurality of treatment protocols. The clinician user interface also comprises a plan modification control configured to modify the plurality of treatment protocols of the treatment plan, and a login interface configured to enable a person to access the clinician user interface by providing a credential associated with one of a plurality of user accounts. Each of the plurality of user accounts has a corresponding set of permissions controlling access to patient data on the clinician user interface.

### BRIEF DESCRIPTION OF THE DRAWINGS

For a detailed description of example embodiments, reference will now be made to the accompanying drawings in which:

FIG. 1 shows a block diagram of an embodiment of a computer implemented system for managing a treatment plan;

FIG. 2 shows a perspective view of an embodiment of a treatment apparatus;

FIG. 3 shows a perspective view of a pedal of the treatment apparatus of FIG. 2;

FIG. 4 shows a perspective view of a person using the treatment apparatus of FIG. 2;

FIG. 5 shows an example embodiment of an overview display of a clinician interface;

FIG. 6 shows an example embodiment of an overview display of a clinician interface with a patient detail overlay;

FIG. 7 shows an example embodiment of an account creation display of a clinician interface;

FIG. 8 shows an example embodiment of a team management display of a clinician interface;

FIG. 9 shows an example embodiment of a team member display of a clinician interface for modifying team member data;

FIG. 10 shows an example embodiment of another team member display of a clinician interface for modifying team member data;

FIG. 11 shows an example embodiment of another team member display of a clinician interface for modifying team member data;

FIG. 12 shows an example embodiment of another team member display of a clinician interface for modifying team member data;

FIG. 13 shows an example embodiment of a notifications settings display of a clinician interface;

FIG. 14 shows an example embodiment of a message center display of a clinician interface;

FIG. 15 shows an example embodiment of a patient data display of a clinician interface for modifying patient personal information;

FIG. 16 shows an example embodiment of another patient data display of a clinician interface for modifying patient personal information;

FIG. 17 shows an example embodiment of a medical data display of a clinician interface for modifying patient procedure details information;

FIG. 18 shows an example embodiment of another medical data display of a clinician interface for modifying patient procedure details information;

FIG. 19 shows an example embodiment of a device data display of a clinician interface;

FIG. 20 shows an example embodiment of a patient profile display of a clinician interface;

FIG. 21 shows another view of the example patient profile display of FIG. 6;

FIG. 22 shows an example embodiment of a treatment protocol management display of a clinician interface;

FIG. 23 shows an example embodiment of a positioning confirmation screen of a patient interface;

FIG. 24 shows an example embodiment of a positioning help screen of a patient interface;

FIG. 25 shows an example embodiment of an adjustment introduction screen of a patient interface;

FIG. 26 shows an example embodiment of an adjustment confirmation screen of a patient interface;

FIG. 27 shows an example embodiment of a session period action screen of a patient interface;

FIG. 28 shows an example embodiment of a first progress data screen of a patient interface.

FIG. 29 shows an example flow diagram of a method for managing a treatment plan.

### NOTATION AND NOMENCLATURE

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

The terminology used herein is for the purpose of describing particular example embodiments only, and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer, or section from another region, layer, or section. Terms such as “first,” “second,” and other numerical terms, when used herein, do not imply a sequence or order unless clearly indicated by the context. Thus, a first element, component, region, layer, or section discussed below could be termed a second element, component, region, layer, or section without departing from the teachings of the example embodiments. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Spatially relative terms, such as “inner,” “outer,” “beneath,” “below,” “lower,” “above,” “upper,” “top,” “bottom,” and the like, may be used herein. These spatially relative terms can be used for ease of description to describe one element's or feature's relationship to another element(s) or feature(s) as illustrated in the figures. The spatially relative terms may also be intended to encompass different orientations of the device in use, or operation, in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptions used herein interpreted accordingly.

### DETAILED DESCRIPTION

The following discussion is directed to various embodiments of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Regulatory frameworks dealing with the practice of medicine have only recently begun to allow telemedicine, or, alternatively a medical practice wherein the patient is virtually seen, diagnosed, prescribed treatments, etc. by a clinician remote from the patient in time and/or physical distance. In light of such recent developments in regulatory frameworks, telemedicine systems using an integrated set of access controls and a privacy enhancing technology (PET) engine executing privacy enhancing technologies to ensure patient privacy have not been addressed heretofore. Telemedicine, including remote physical therapy, presents a number of new challenges for protecting patient data and for compliance with patient privacy regulations such as the Health Insurance Portability and Accountability Act (HIPAA), the California Consumer Privacy Act (CCPA), or the General Data Protection Regulation (GDPR). Specifically, the present disclosure provides a unique combination of computerized access controls with PETs to enable a novel system 10 engineered to provide information for practicing remote physical therapy by a patient separated from a supervising clinician in time and/or physical distance. More specifically, the system 10 of the present disclosure incorporates a PET engine and user access controls to provide information tailored for use by different individuals, including clinicians, patients, practice managers, and staff members. The system 10 of the present disclosure, thereby, enables remote physical therapy, via a telemedicine session between one or more computing devices (e.g., a patient, a clinician, practice manager, staff members, etc.) compliant with privacy regulations and other regulatory frameworks that pertain to the practice of medicine.

In some embodiments, the PET engine may be implemented in computer instructions stored in one or more memory devices of one or more servers and executed by one or more processing devices of the one or more servers. The one or more servers may be included in distributed, cloud-based computing system. The PET engine may execute one or more machine learning models trained to use one or more privacy enhancing technologies (PETs) to control access to PII. The machine learning models may be trained with training data that maps inputs with certain outputs, such that the machine learning models are trained to identify pattern in data that should be de-identified, re-identified, protected, removed, stored, modified, etc. The training of the machine learning models may be iteratively performed to dynamically change based on certain regulations, laws, and/or protocols that are enacted or de-enacted. Accordingly, the PET engine enables dynamically learning and maintaining up-to-date machine learning models based on the current regulations, laws, and/or protocols pertaining to data privacy.

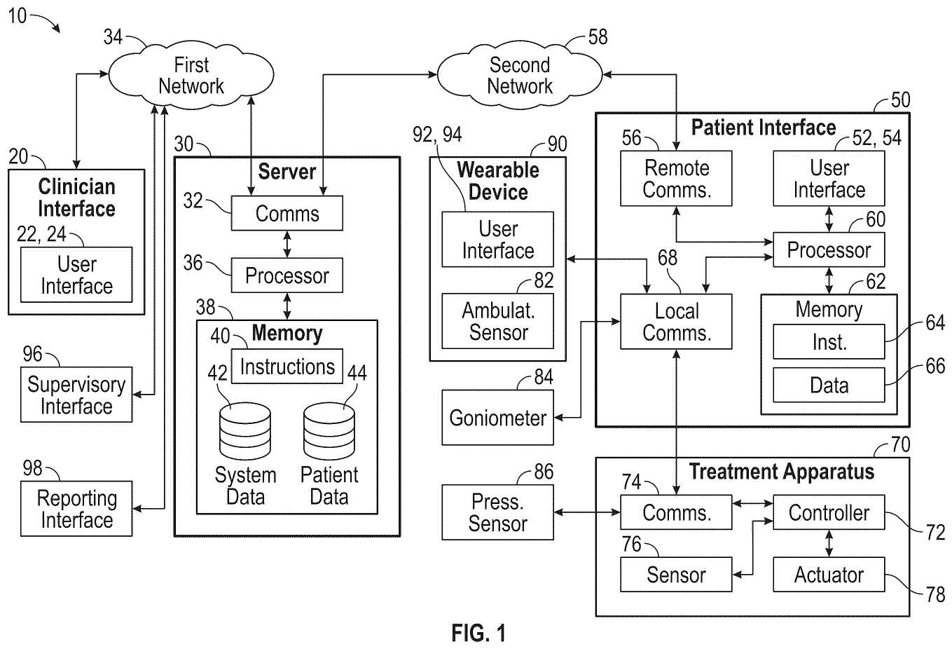

FIG. 1 shows a block diagram of a computer-implemented system 10, hereinafter called “the system” for managing a treatment plan. The treatment plan includes one or more treatment protocols, and each treatment protocol includes one or more sessions. Each session comprises several session periods, with each session period including a particular activity for treating the body part of the patient. For example, a treatment plan for post-operative rehabilitation after a knee surgery may include an initial treatment protocol with twice daily stretching sessions for the first 3 days after surgery and a more intensive treatment protocol with active exercise sessions performed 4 times per day starting 4 days after surgery.

The system 10 includes a clinician interface 20 for a clinician, such as a doctor, a nurse, a physical therapist, or a technician, to use to review and to configure various aspects of a treatment plan for use in treating a patient. The clinician interface 20 includes a clinician input device 22 and a clinician display 24, which may be collectively called a clinician user interface 22, 24. The clinician input device 22 may include one or more of a keyboard, a mouse, a trackpad, or a touch screen, for example. Alternatively or additionally, the clinician input device 22 may include one or more microphones and voice-based functionalities, with hardware and/or software configured to interpret spoken instructions by the clinician by using the one or more microphones. The clinician input device 22 may include functionality provided by or similar to existing voice-based assistants such as Siri by Apple, Alexa by Amazon, Google Assistant, or Bixby by Samsung. The clinician input device 22 may include other hardware and/or software components. The clinician input device 22 may include one or more general purpose devices and/or special-purpose devices.

The clinician display 24 may take one or more different forms including, for example, a computer monitor or display screen on a tablet, smartphone, or a smart watch. The clinician display 24 may include other hardware and/or software components such as a projector, virtual reality capability, or augmented reality capability etc. The clinician display 24 may incorporate various different visual, audio, or other presentation technologies. For example, the clinician display 24 may include a non-visual display, such as an audio signal, which may include spoken language and/or other sounds such as tones, chimes, and/or melodies which may signal different conditions and/or directions. The clinician display 24 may comprise one or more different display screens presenting various data and/or interfaces or controls for use by the clinician. The clinician display 24 may include graphics, which may be presented by a web-based interface and/or by a computer program or application (App.).

The system 10 also includes a server 30 configured to store and to provide data related to managing the treatment plan. The server 30 may include one or more computers and may take the form of a distributed and/or virtualized computer or computers. In some embodiments, the server 30 may generate aspects of the clinician display 24 for presentation by the clinician interface 20. For example, the server 30 may include a web server configured to generate the display screens for presentation upon the clinician display 24. In some embodiments, the clinician display 24 may be configured to present a virtualized desktop that is hosted by the server 30. The server 30 also includes a first communication interface 32 configured to communicate with the clinician interface 20 via a first network 34. In some embodiments, the first network 34 may include a local area network (LAN), such as an Ethernet network. In some embodiments, the first network 34 may include the Internet, and communications between the server 30 and the clinician interface 20 may be secured via encryption, such as, for example, by using a virtual private network (VPN). In some embodiments, the first network 34 may include wired and/or wireless network connections such as Wi-Fi, Bluetooth, ZigBee, Near-Field Communications (NFC), cellular data network, etc. The server 30 includes a first processor 36 and a first machine-readable storage memory 38, which may be called a “memory” for short, holding first instructions 40 for performing the various actions of the server 30 for execution by the first processor 36.

The server 30 is configured to store data regarding the treatment plan. For example, the memory 38 includes a system data store 42 configured to hold system data, such as data pertaining to treatment plans for treating one or more patients. The server 30 is also configured to store data regarding performance by a patient in following a treatment plan. For example, the memory 38 includes a patient data store 44 configured to hold patient data, such as data pertaining to the one or more patients, including data representing each patient's performance within the treatment plan.

The server 30 is configured to execute and be controlled by a privacy-enhancing technology (PET) engine that uses one or more privacy-enhancing technologies that control access to personally identifiable information (PII) associated with the patient. Controlling access may refer to defining access, enabling access, disabling access, etc. In some embodiments, the PET engine is configured to pseudonymize or anonymize the PII associated with the patient. In some embodiments, the PET engine may enable de-identification and/or re-identification of the PII associated with the patient. PETs, as used by the PET engine herein, may include, without limitation, differential privacy, homomorphic encryption, public key encryption, digital notarization, pseudonymization, pseudonymisation, anonymization, anonymisation, digital rights management, k-anonymity, I-diversity, synthetic data creation, suppression, generalization, identity management, and the introduction of noise into existing data or systems.

In some embodiments, the computer-implemented system 10 is configured to maintain a plurality of user accounts, with each of the user accounts having an account type associated therewith, and each of the plurality of user accounts has a corresponding set of permissions enabling an owner of the user account to access the patient data. The computer-implemented system 10 may be configured to restrict access to each of the user accounts using login credentials.

The account types may include a super-administrator account type. A user account having the super-administrator account type may have unrestricted access to the patient data, or at least a greater access to the patient data than any other one of the account types. A user having the super-administrator account type can also be a controlling entity with respect to the granting or revocation of access to the PII or any portion thereof and with respect to which other entities are granted or denied such access, including the level of such access granted or denied, and, last with respect to any other conditions, such as time, location, identity, etc. which may further be used by a PET engine to grant or deny any of the types of access contemplated.

The account types may also include a practice manager account type. In some embodiments, the practice manager may have access to all patient data for patients assigned to the practice manager. In some embodiments, the practice manager may have access to view and/or to modify at least some of the patient data for patients assigned to the practice manager. More specifically, the practice manager may have access to some, but not all, patient data for patients assigned to the practice manager. For example, the practice manager may have access to biographical and/or scheduling data within the patient data, but the practice manager may not have access to view and/or modify to certain types of medical data within the patient data. In some embodiments, the practice manager may not have any access to patient data. For example, the practice manager may be limited to managing user accounts of other team members, such as clinicians and/or staff members.

The account types may also include a clinician account type. A user account having the clinician account type may have access to view and/or to modify at least some of the patient data for patients assigned to the owner of the user account having the clinician account type. For example, a particular doctor may have a user account with the clinician account type. The particular doctor may have access to view and modify patient data of patients that are assigned to that particular doctor. However, the particular doctor may not have access to patient data regarding other patients that are not assigned to that particular doctor.

The account types may also include a staff member account type. The staff member account type may be used, for example, by a nurse or a physical therapist. A user account having the staff member account type may have access to view and/or to modify at least some of the patient data for patients assigned to the owner of the user account having the staff member account type. For example, a particular nurse may have a user account with the staff member account type. The particular nurse may have access to view and modify patient data of patients assigned to that particular nurse. However, the particular doctor may not have access to patient data regarding other patients not assigned to that particular nurse.

The account types may also include a patient account type. A user account having the patient account type may have access to view at least some of the patient data associated with their own user account. However, the user account having the patient account type may not have access to modify any patient data.

In some embodiments, user accounts having one or more of the account types may have abilities to designate another one of the user accounts as having predetermined account types. For example, a user account having the practice manager account type may have the ability to designate another user account as having the clinician account type. Additionally or alternatively, the user account having the practice manager account type may have the ability to designate another user account as having the staff member or patient user type. In another example, a user account having the clinician account type may have the ability to designate another user account as having the staff member account type. Additionally or alternatively, the user account having the clinician account type may have the ability to designate another user account as having the patient user type.

The system 10 also includes a patient interface 50 configured to communicate information to a patient and to receive feedback from the patient. Specifically, the patient interface 50 includes an input device 52 and an output device 54, which may be collectively called a patient user interface 52, 54. The input device 52 may include one or more devices, such as a keyboard, a mouse, a touch screen input, a gesture sensor, and/or a microphone and processor configured for voice recognition. The output device 54 may take one or more different forms including, for example, a computer monitor or display screen on a tablet, smartphone, or a smart watch. The output device 54 may include other hardware and/or software components such as a projector, virtual reality capability, augmented reality capability, etc. The output device 54 may incorporate various different visual, audio, or other presentation technologies. For example, the output device 54 may include a non-visual display, such as an audio signal, which may include spoken language and/or other sounds such as tones, chimes, and/or melodies, which may signal different conditions and/or directions. The output device 54 may comprise one or more different display screens presenting various data and/or interfaces or controls for use by the patient. The output device 54 may include graphics, which may be presented by a web-based interface and/or by a computer program or application (App.).

As shown in FIG. 1, the patient interface 50 includes a second communication interface 56, which may also be called a remote communication interface configured to communicate with the server 30 and/or the clinician interface 20 via a second network 58. In some embodiments, the second network 58 may include a local area network (LAN), such as an Ethernet network. In some embodiments, the second network 58 may include the Internet, and communications between the patient interface 50 and the server 30 and/or the clinician interface 20 may be secured via encryption, such as, for example, by using a virtual private network (VPN). In some embodiments, the second network 58 may include wired and/or wireless network connections such as Wi-Fi, Bluetooth, ZigBee, Near-Field Communications (NFC), cellular data network, etc. In some embodiments, the second network 58 may be the same as and/or operationally coupled to the first network 34.

The patient interface 50 includes a second processor 60 and a second machine-readable storage memory 62 holding second instructions 64 for execution by the second processor 60 for performing various actions of patient interface 50. The second machine-readable storage memory 62 also includes a local data store 66 configured to hold data, such as data pertaining to a treatment plan and/or patient data, such as data representing a patient's performance within a treatment plan. The patient interface 50 also includes a local communication interface 68 configured to communicate with various devices for use by the patient in the vicinity of the patient interface 50. The local communication interface 68 may include wired and/or wireless communications. In some embodiments, the local communication interface 68 may include a local wireless network such as Wi-Fi, Bluetooth, ZigBee, Near-Field Communications (NFC), cellular data network, etc.

The system 10 also includes a treatment apparatus 70 configured to be manipulated by the patient and/or to manipulate a body part of the patient for performing activities according to the treatment plan. In some embodiments, the treatment apparatus 70 may take the form of an exercise and rehabilitation apparatus configured to perform and/or to aid in the performance of a rehabilitation regimen, which may be an orthopedic rehabilitation regimen, and the treatment includes rehabilitation of a body part of the patient, such as a joint or a bone or a muscle group. More specifically, the regimen may be a physical rehabilitation regimen for improving strength and/or range of motion of the body part. The body part may include, for example, a spine, a hand, a foot, a knee, or a shoulder. The body part may include a part of a joint, a bone, or a muscle group, such as one or more vertebrae or a ligament. As shown in FIG. 1, the treatment apparatus 70 includes a controller 72, which may include one or more processors, computer memory, and/or other components. The treatment apparatus 70 also includes a fourth communication interface 74 configured to communicate with the patient interface 50 via the local communication interface 68. The treatment apparatus 70 also includes one or more internal sensors 76 and an actuator 78, such as a motor. The actuator 78 may be used, for example, for moving the patient's body part and/or for resisting forces by the patient.

The internal sensors 76 may measure one or more operating characteristics of the treatment apparatus 70 such as, for example, a force a position, a speed, and/or a velocity. In some embodiments, the internal sensors 76 may include a position sensor configured to measure at least one of a linear motion or an angular motion of a body part of the patient. For example, an internal sensor 76 in the form of a position sensor may measure a distance that the patient is able to move a part of the treatment apparatus 70, where such distance may correspond to a range of motion that the patient's body part is able to achieve. In some embodiments, the internal sensors 76 may include a force sensor configured to measure a force applied by the patient. For example, an internal sensor 76 in the form of a force sensor may measure a force or weight the patient is able to apply, using a particular body part, to the treatment apparatus 70.

The system 10 shown in FIG. 1 also includes an ambulation sensor 82, which communicates with the server 30 via the local communication interface 68 of the patient interface 50. The ambulation sensor 82 may track and store a number of steps taken by the patient. In some embodiments, the ambulation sensor 82 may take the form of a wristband, wristwatch, or smart watch. In some embodiments, the ambulation sensor 82 may be integrated within a phone, such as a smartphone.

The system 10 shown in FIG. 1 also includes a goniometer 84, which communicates with the server 30 via the local communication interface 68 of the patient interface 50. The goniometer 84 measures a position of the patient's body part. More specifically, the goniometer 84 measures an angle of the body part, particularly where the body part is a joint. For example, the goniometer 84 may measure the angle of flex of a patient's knee or elbow or shoulder.

The system 10 shown in FIG. 1 also includes a pressure sensor 86, which communicates with the server 30 via the local communication interface 68 of the patient interface 50. The pressure sensor 86 measures an amount of pressure or weight applied by a body part of the patient. For example, pressure sensor 86 may measure an amount of force applied by a patient's foot when pedaling a stationary bike.

The system 10 also includes a wearable device 90 configured to be worn or carried on the patient's person. The wearable device 90 may take one of several different forms such as, for example, a smart watch, a wristband, a pendant, or a smartphone. The wearable device 90 may include a means of attachment, such as a pin, a belt clip, a strap, or a lanyard, to facilitate the device's being worn or carried by the patient. In some embodiments, and as shown in FIG. 1, the wearable device 90 includes the ambulation sensor 82. The wearable device 90 may include one or more other sensors, such as a heartrate sensor, a blood pressure sensor, or a pulse oximeter. The ambulation sensor 82 or another one of the sensors in the wearable device 90 may be configured to monitor one or more factors that indicate an activity level of the patient. The patient's activity level could be used to determine a quantity and/or quality of exercise performed by the patient. The patient's activity level could also be used to determine a quantity and/or quality of the patient's sleep.

The wearable device 90 includes a wearable input device 92 and a wearable display 94, which may be collectively called a wearable user interface 92, 94. The wearable input device 92 may include one or more devices, such as a keyboard, a mouse, a touch screen input, a gesture sensor, and/or a microphone and processor configured for voice recognition. The wearable display 94 may take one or more different forms including, for example, a display screen, and/or one or more lights or other indicators. The wearable display 94 may incorporate various different visual, audio, or other presentation technologies. For example, the wearable display 94 may include a non-visual display, such as a haptic or tactile device and/or an audio signal, which may include spoken language and/or other sounds such as tones, chimes, and/or melodies, and the non-visual display may signal different conditions and/or directions. The wearable display 94 may comprise one or more different display screens configured to present various data and/or interfaces or controls for use by the patient. The wearable display 94 may include graphics, which may be presented by a web-based interface and/or by a computer program or application (App.). The wearable user interface 92, 94 may be configured to present different types of information to the patient. For example, the wearable user interface 92, 94 may be configured to present a reminder when it is time for the patient to perform a rehabilitation session. The wearable user interface 92, 94 may allow the patient to track daily goals or to receive messages from a clinician, etc. This function of the wearable device 90 may be especially useful when the patient is away from the patient interface 50.

The system 10 shown in FIG. 1 also includes a supervisory interface 96 which may be similar or identical to the clinician interface 20. In some embodiments, the supervisory interface 96 may have enhanced functionality beyond what is provided on the clinician interface 20. The supervisory interface 96 may be configured for use by a person having responsibility for the treatment plan, such as an orthopedic surgeon.

The system 10 shown in FIG. 1 also includes a reporting interface 98 which may be similar or identical to the clinician interface 20. In some embodiments, the reporting interface 98 may have less functionality from what is provided on the clinician interface 20. For example, the reporting interface 98 may not have the ability to modify a treatment plan. Such a reporting interface 98 may be used, for example, by a biller to determine the use of the system 10 for billing purposes. In another example, the reporting interface 98 may not have the ability to display patient identifiable information, presenting only pseudonymized data and/or anonymized data for certain data fields concerning a data subject and/or for certain data fields concerning a quasi-identifier of the data subject. Such a reporting interface 98 may be used, for example, by a researcher to determine various effects of a treatment plan on different patients.

In some embodiments, the patient interface 50 and the treatment apparatus 70 are each configured to operate from a patient location geographically separate from a location of the clinician interface 20. For example, the patient interface 50 and the treatment apparatus 70 may be used as part of an in-home rehabilitation system, which may be monitored remotely by using the clinician interface 20 at a centralized location, such as a clinic or hospital. In some embodiments, either or both of the patient interface 50 and/or the treatment apparatus 70 are configured to communicate with a remote computer, such as the server 30, to receive the treatment plan and to report back to the remote computer with data regarding performance by the patient in following the treatment plan.

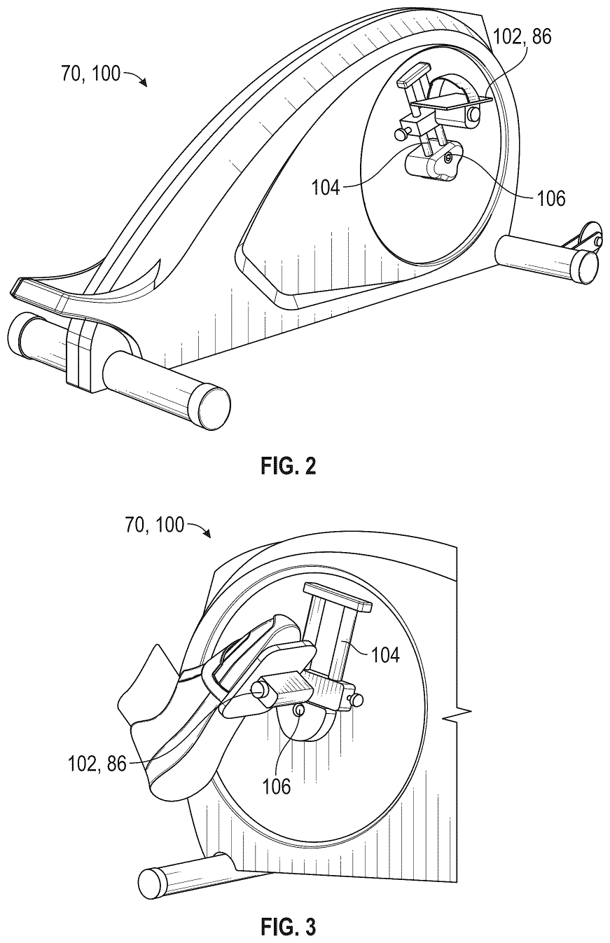

FIGS. 2-3 show an embodiment of a treatment apparatus 70. More specifically, FIG. 2 shows a treatment apparatus 70 in the form of a stationary cycling machine 100, which may be called a stationary bike, for short. The stationary cycling machine 100 includes a set of pedals 102 each attached to a pedal arm 104 for rotation about an axle 106. In some embodiments, and as shown in FIG. 2, the pedals 102 are movable on the pedal arms 104 in order to adjust a range of motion used by the patient in pedaling. For example, the pedals being located inwardly toward the axle 106 corresponds to a smaller range of motion than when the pedals are located outwardly away from the axle 106. A pressure sensor 86 is attached to or embedded within one of the pedals 106 for measuring an amount of force applied by the patient on the pedal 106. The pressure sensor 86 may communicate wirelessly to the treatment apparatus 70 and/or to the patient interface 50.

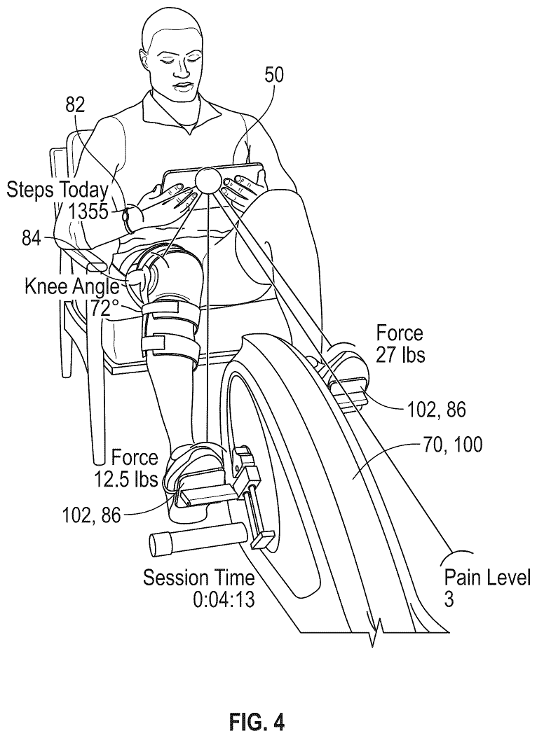

FIG. 4 shows a person (a patient) using the treatment apparatus of FIG. 2, and showing sensors and various data parameters connected to a patient interface 50. The example patient interface 50 is a tablet computer or smartphone, or a phablet, such as an iPad, an iPhone, an Android device, or a Surface tablet, which is held manually by the patient. In some other embodiments, the patient interface 50 may be embedded within or attached to the treatment apparatus 70. FIG. 4 shows the patient wearing the ambulation sensor 82 on his wrist, with a note showing “STEPS TODAY 1355”, indicating that the ambulation sensor 82 has recorded and transmitted that step count to the patient interface 50. FIG. 4 also shows the patient wearing the goniometer 84 on his right knee, with a note showing “KNEE ANGLE 72°”, indicating that the goniometer 84 is measuring and transmitting that knee angle to the patient interface 50. FIG. 4 also shows a right side of one of the pedals 106 with a pressure sensor 86 showing “FORCE 12.5 lbs.,” indicating that the right pedal pressure sensor 86 is measuring and transmitting that force measurement to the patient interface 50. FIG. 4 also shows a left side of one of the pedals 106 with a pressure sensor 86 showing “FORCE 27 lbs.”, indicating that the left pedal pressure sensor 86 is measuring and transmitting that force measurement to the patient interface 50. FIG. 4 also shows other patient data, such as an indicator of “SESSION TIME 0:04:13”, indicating that the patient has been using the treatment apparatus 70 for 4 minutes and 13 seconds. This session time may be determined by the patient interface 50 based on information received from the treatment apparatus 70. FIG. 4 also shows an indicator showing “PAIN LEVEL 3”. Such a pain level may be obtained from the patent in response to a solicitation, such as a question, presented upon the patient interface 50.

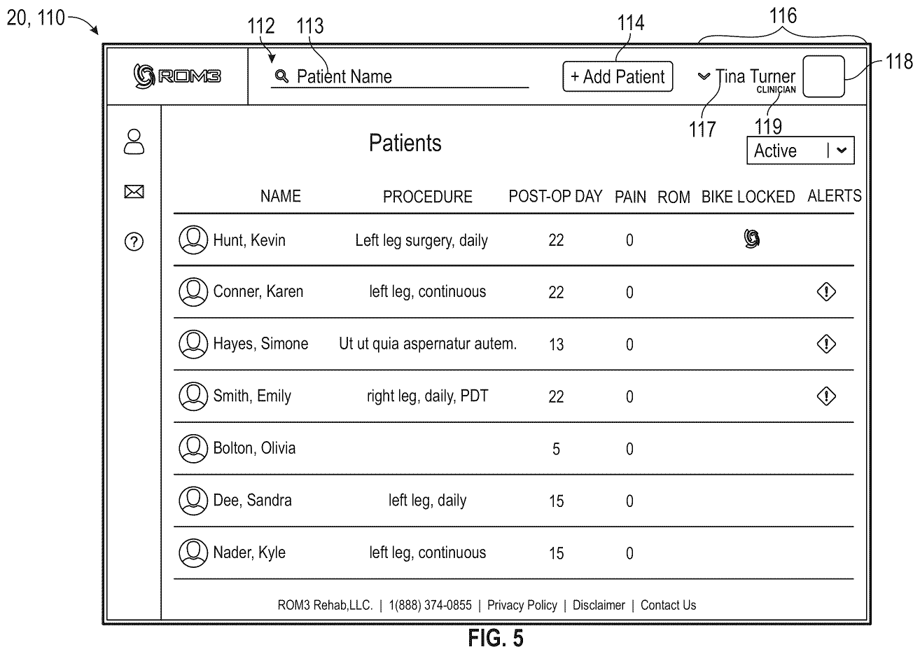

FIG. 5 is an example embodiment of an overview display 110 of the clinician interface 20. Specifically, the overview display 110 presents summary information regarding each of a plurality of different patients. In some embodiments, and as shown on FIG. 5, the summary information includes an indicator showing a procedure performed upon each of the patients, temporal progress of the patient within the treatment plan (post-op day), an indicator of a last-reported pain level, range-of-motion (ROM) numbers, and an indicator showing if there are any alerts requiring special attention.

FIG. 5 also shows a header 112, which is shared with many or all of the other screens of the clinician interface 20. The header 112 includes a patient search input 113 whereby a user may quickly access patient data by entering the patient's name. The header 112 also includes an Add Patient control 114 in the form of a button. When pressed, the Add Patient control 114 causes the clinician interface 20 to enable a user to add a new patient to the system 10. The header 112 also includes a user account login control 116 presenting information regarding the user account currently logged-in to the clinician interface 20, and allowing a user to change the user account logged-in to the clinician interface 20. As shown in the example display on FIG. 5, the user account login control 116 includes a name 117 and a pictorial icon 118 associated with the user account currently logged-in to the clinician interface 20. The user account login control 118 also includes an account type indicator 119 of the account type (e.g., Clinician, Staff, Practice Manager, etc.) for the user account currently logged-in to the clinician interface 20.

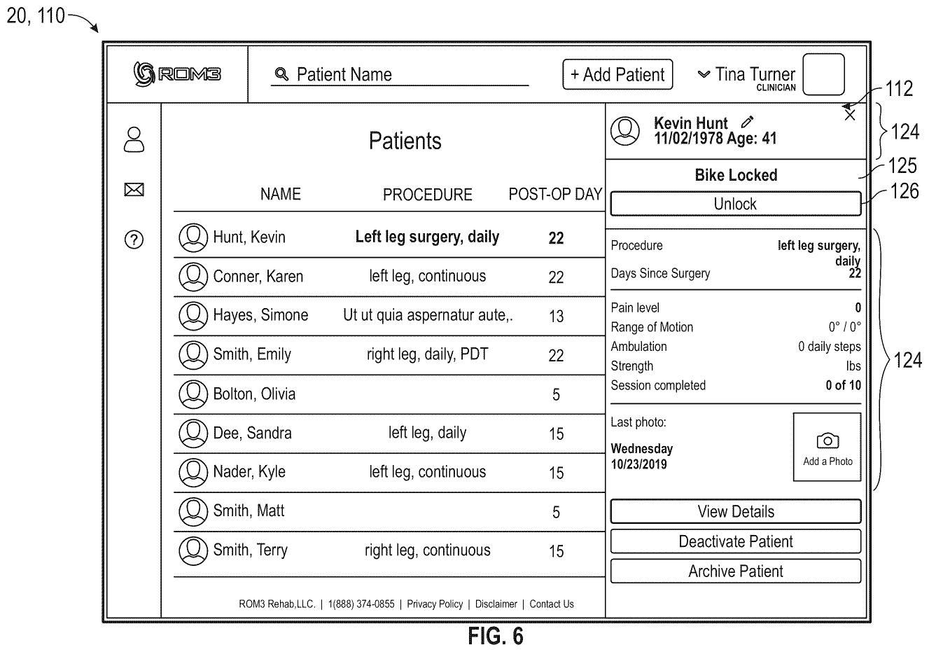

FIG. 6 shows an example embodiment of the overview display 110 of the clinician interface 20 with a patient detail overlay 122. The patient detail overlay 122 may take the form of a modal window or a popup window that overlies the overview display 120. Alternatively, the patient detail overlay 122 may be a separate display or screen on the clinician interface 20. In some embodiments, the patient detail overlay 122 may be presented in response to a user of the clinician interface 20 selecting one of the patients listed on the overview display 120. Alternatively or additionally, the patient detail overlay 122 for a particular patient may be presented in response to an occurrence of a triggering condition regarding that particular patient, such as the particular patient's treatment apparatus 70 being in a locked condition. The patient detail overlay 122 includes several patient profile data fields 124, each presenting corresponding data regarding the patient. The data patient profile data fields 124 of the patient detail overlay 122 may include, for example, name, age, date of birth, procedure for which the patient is being treated, days since surgery, last reported pain level, range of motion statistics, ambulation data, strength data, sessions completed within the treatment plan, a photograph of the patient, etc. The patient detail overlay 122 also includes an apparatus status display 125 and an apparatus unlock control 124 for a user of the clinician interface 20 to unlock or to re-enable the treatment apparatus 70.

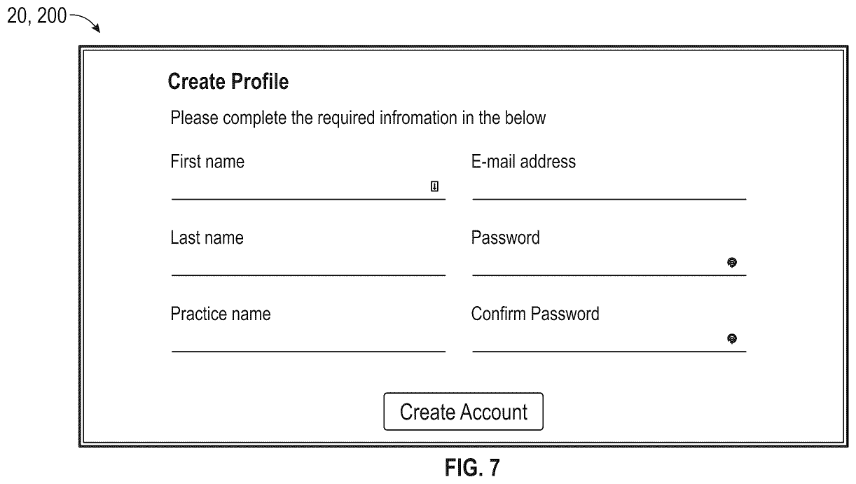

FIG. 7 shows an example embodiment of an account creation display 200 of the clinician interface 20. The example account creation display 200 includes fields for data entry for use in creating a user account in the system 10.

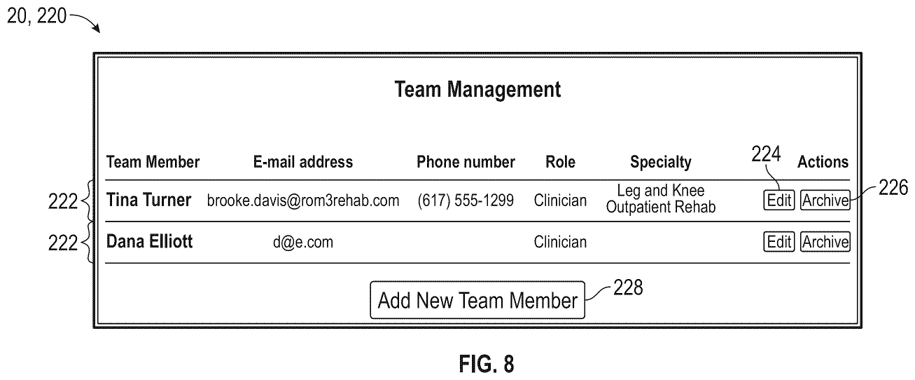

FIG. 8 shows an example embodiment of a team management display 220 of the clinician interface 20. The team management display 220 presents a list of user accounts associated with a practice. The example team management display 220 includes team member records 222 for each of a plurality of user accounts having an account type associated with team members (e.g., clinician or staff member) who have access to the patient data by virtue of their position. For example, the team members may all be individuals that are clinicians or staff members of a particular practice managed by a practice manager. The team management display 220 may present the team member records 222 in rows or in other forms, such as tabular data, graphical icons, etc. As shown in the example team management display 220 of FIG. 8, the team member records 222 each include summary biographical information such as name, phone number, role (i.e., clinician or staff member), and specialty. The example team management display 220 includes an edit control 224, such as a button, associated with each of the team member records 222 for enabling a user to modify attributes of the associated team member. The example team management display 220 also includes an archive control 226, such as a button, associated with each of the team member records 222, for enabling a user to archive or to disable the associated team member. The edit control 224 and/or the archive control 226 may be available only for users having certain user types. For example, a user having the clinician user type may have the ability to modify or to archive accounts having the staff member user type, but the user having the clinician user type may not have the ability to modify or to archive user accounts having user accounts that have the clinician user type. The example team management display 220 also includes a new user add control 228, which may take the form of a button, configured to add a new user account or to associate an account type with a new or an existing user account.

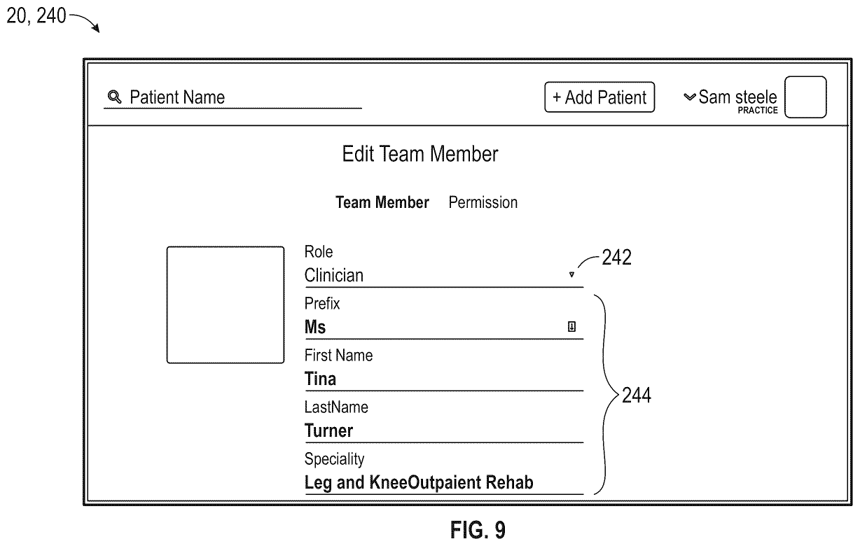

FIG. 9 shows an example embodiment of a first team member display 240 of the clinician interface 20 for modifying team member data. Using an edit control 224 on the team management display 220, the first team member display 240 may be invoked for a selected team member. The example first team member display 240 presents an account type control input 242 for modifying an account type associated with the selected user account. The account type control input 242 is shown in FIG. 9 as being a drop-down selector type control, although the account type control input 242 may take any one of several different forms including radio buttons or a text entry field. The example first team member display 240 also presents other data entry fields 244 for a user to input various data regarding a team member. The data entry fields 244 may include, for example, inputs for name and specialty.

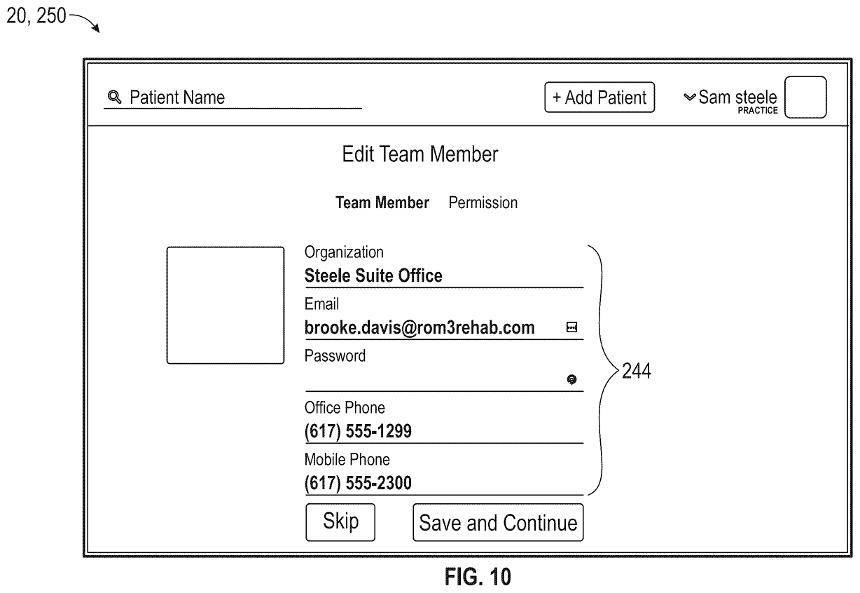

FIG. 10 shows an example embodiment of a second team member display 250 of the clinician interface 20 for modifying team member data. The second team member display 250 may be a continuation of the first team member display 240. The example second team member display 250 includes additional data entry fields 244 for additional data regarding the team member, such as organization, email address, and phone numbers. The example second team member display 250 also includes a skip button 246 for allowing a user to bypass entering information into the data entry fields 244. The example second team member display 250 also includes a “save and continue” button 248 for saving information entered into the data entry fields 244 to the system data store 42 for association with the user account of the selected team member.

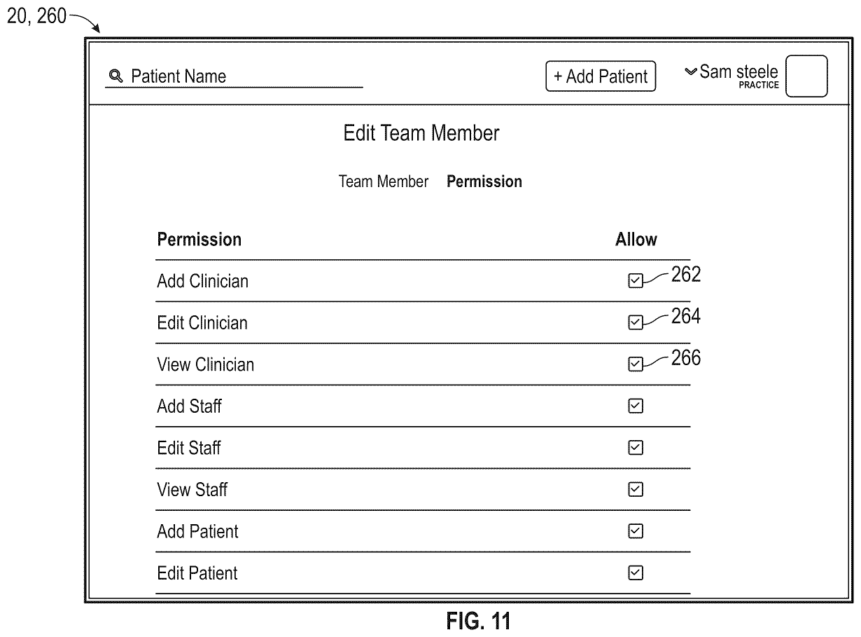

FIG. 11 shows an example embodiment of a third team member display 260 of the clinician interface 20 for modifying team member data. The third team member display 260 presents a plurality of permission controls, with each of the permission controls configured to modify an ability of the selected user account to perform an action or to view or to modify a subset of the patient data. Specifically, the third team member display 260 includes an “add clinician” permission control 262 in the form of a check box. The “add clinician” permission control 262 enables the selected user account to add a user account having the clinician account type or to designate another one of the user accounts as the clinician account type.

The third team member display 260 also includes a plurality of account permission controls associated with a given account type. For example, the account permission controls include an “edit clinician” permission control 264 in the form of a check box. The “edit clinician” permission control 264 enables the selected user account to modify a user account having the clinician account type. The account permission controls also include a “view clinician” permission control 266 in the form of a check box. The “view clinician” permission control 266 enables the selected user account to view one or more non-public attributes of a user account having the clinician account type. The account permission controls 262, 264, 266 may take other forms such as, for example, radio buttons or a drop-down selector. The third team member display 260 includes add/edit/view account permission controls (not labeled) for other account types including staff member and patient.

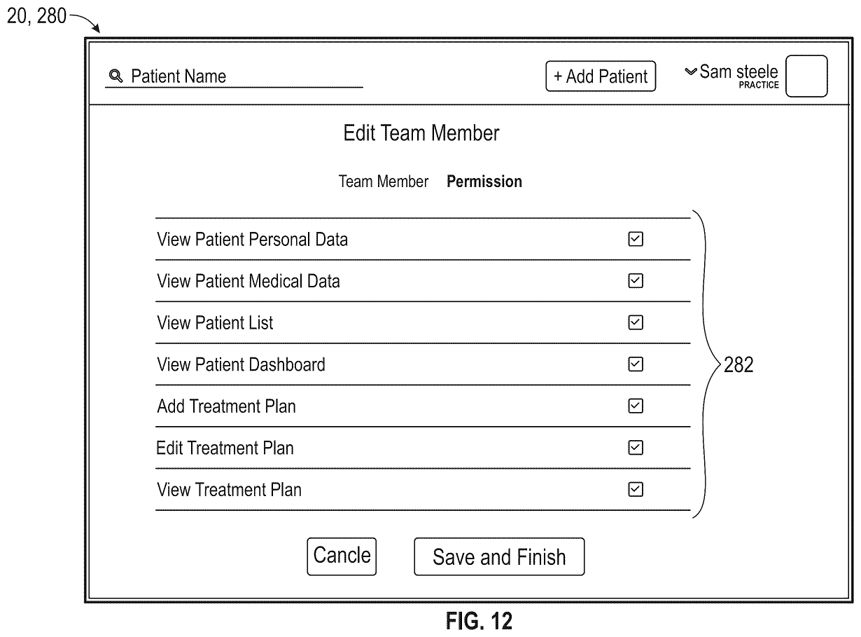

FIG. 12 shows an example embodiment of a fourth team member display 280 of the clinician interface 20 for modifying team member data. Specifically, the example fourth team member display 280 includes a plurality of patient data permission controls 282, with each of the patient data permission controls 282 configured to modify an ability of the selected user account to perform an action or to view or to modify a subset of the patient data. For example, the permission controls 282 include check boxes for selectively allowing the selected user account to view patient personal information, patient medical data, the patient list, the patient dashboard, and/or to add, edit, or to view patient treatment plans. The patient data permission controls 282 may take other forms such as, for example, radio buttons, or drop-down selectors. As used herein, “patient personal information” refers to a bibliographic subset of PII, e.g., name, address, email, etc., but not medical data, conditions, measurements, etc.”

The team member displays 240, 250, 260, 208 of FIGS. 9-12 may have other configurations or arrangements. For example, the team member modification displays 240, 250, 260 may be combined as any of one or more display screens.

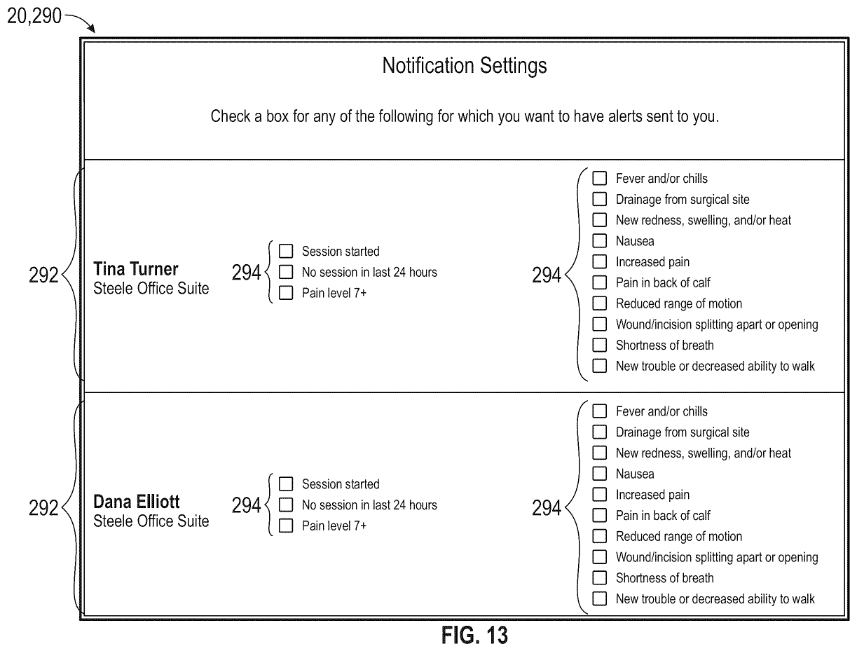

FIG. 13 shows an example embodiment of a notifications settings display 290 of the clinician interface 20. The example notifications settings display 290 includes a notification control panel 294 for each of a plurality of user accounts having an account type associated with team members (e.g. clinician or staff member) who have access to the patient data by virtue of their position. The notifications settings display 290 may present the notification control panel 292 in rows or in other forms, such as tabular data, graphical icons, etc. As shown in the example notifications settings display 290 of FIG. 13, the notification control panels 292 each include a plurality of notification enable controls 294. Each of the notification enable controls 294 is configured for designating an associated trigger condition as being enabled or disabled for a specified user account (i.e., the user account associated with the notification control panel 292 that contains the notification enable control 294). Various different trigger conditions having notification enable controls 294 are shown on FIG. 13. Those trigger conditions may include, for example, a patient starting a treatment session, a patient reporting a pain level of 7 or greater, or a reduction in range of motion. The notification enable controls 294 are shown on FIG. 13 as check boxes, however, the notification enable controls 294 may take other forms such as, for example, radio buttons or a drop-down selector.

The system 10 is configured to send an alert message to a person having the specified user account in response to occurrence of a given trigger condition for any patients assigned to the person having the specified user account, but only if the given trigger condition is designated as being enabled for the specified user account. The notification trigger event may be a reported event, which is an event based upon a condition reported by the patient, such as a pain level, a report of nausea or wound/incision splitting. Alternatively or additionally, the trigger event may be a measured event resulting from satisfaction of a condition that includes a measurement of the patient. Measured events may include, for example, a heartrate measured by a heartrate monitor exceeding a predetermined value or reduced range of motion, as determined by a goniometer 84 which performed measurements of a patient's knee.

In some embodiments, the alert message may be sent using a communication channel outside of the clinician user interface 20 to the person having the specified user account. For example, the system 10 may be configured to send the alert message in the form of a phone call, an email, a text message, or a pager message. In some embodiments, the alert message does not include any unacceptable PII regarding the patient. For example, the alert message may include a generic message directing the person to check the clinician user interface 20. The alert message may include some additional information, such as a type of trigger condition or a severity of an associated condition (e.g., critical alert or non-critical warning alert) without including unacceptable PII regarding the patient.

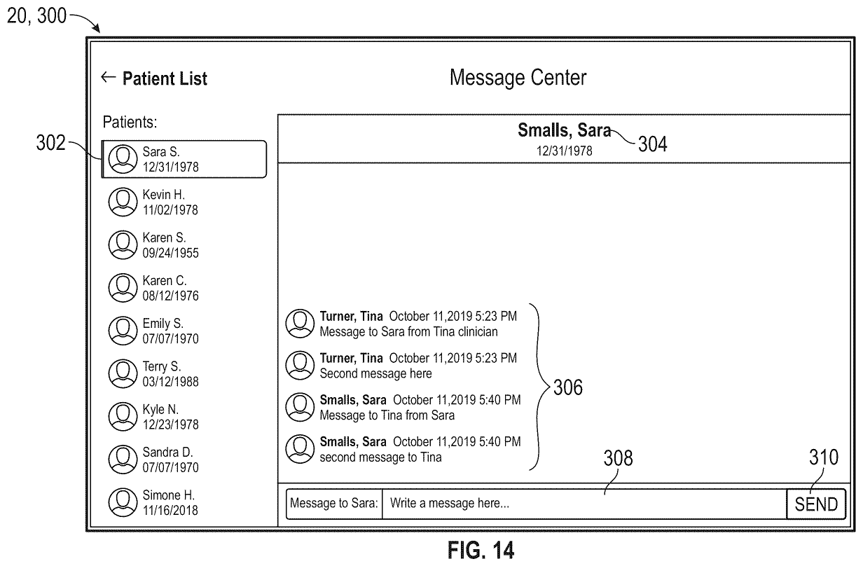

FIG. 14 shows an example embodiment of a message center interface 300 of the clinician interface 20. The message center interface 300 may be configured as a display screen 300 as shown in FIG. 14, although the message center interface 300 may have other forms or appearances. The message center interface 300 configured to enable a textual message exchange between owners of different user accounts. Specifically, the example message center interface 300 includes a patient selector 302 for a user to select one of a plurality of patients to communicate with. The message center interface 300 also includes a selected patient indicator 304 that shows identifying information regarding a patient currently selected to send, receive, and/or view messages. The identifying information in the selected patient indicator 304 includes name and date of birth of the selected patient. The message center interface 300 also includes a message history display 306 showing a record of one or more previous textual messages exchanged between the person using the message center interface 300 and the selected patient. The message center interface 300 also includes a message input field 308 for the person using the message center interface 300 (e.g., a clinician or staff member) to compose textual messages to be sent to the selected patient). The message center interface 300 also includes a message send control 310, such as a button, and which is configured to cause the system 10 to deliver the textual message to the patient.

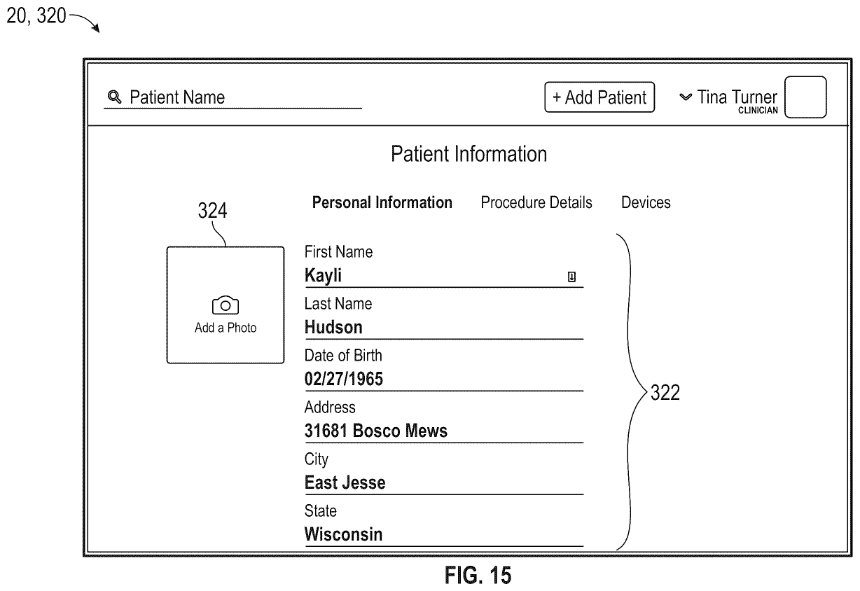

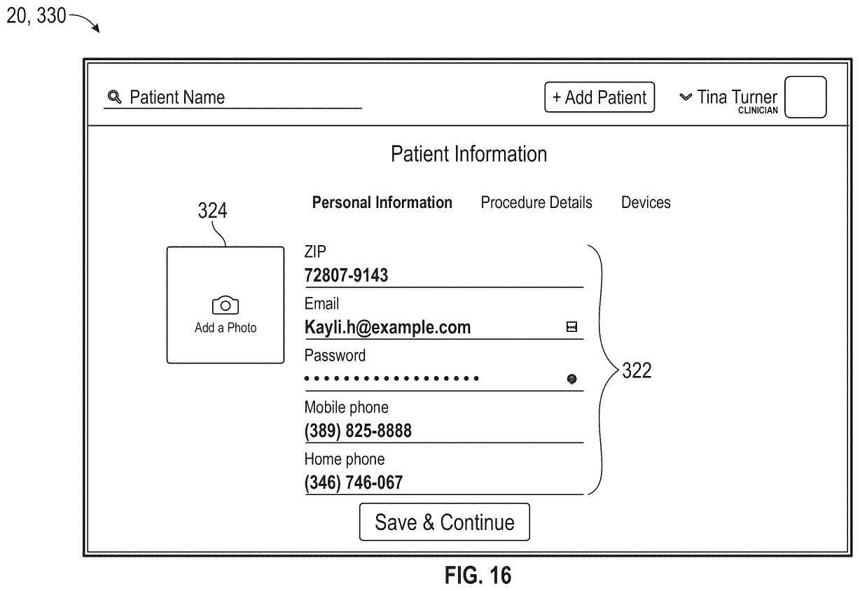

FIGS. 15-16 show example embodiments of patient data displays 320, 330 of the clinician interface 20. More specifically, the example patient data displays 320, 330 of FIGS. 15-16 are configured for modifying personal information regarding a patient. The patient data displays 320, 330 may be presented using one or more screens or displays. Each of the patient data displays 320, 330 includes a plurality of PII fields 322, each holding a corresponding PII item regarding a given patient. The PII items may include, for example, first name, last name, date of birth, email address, mobile phone number, etc. Any or all of the patient data displays 320, 330 may include a photo display 324 showing a picture of the given patient.

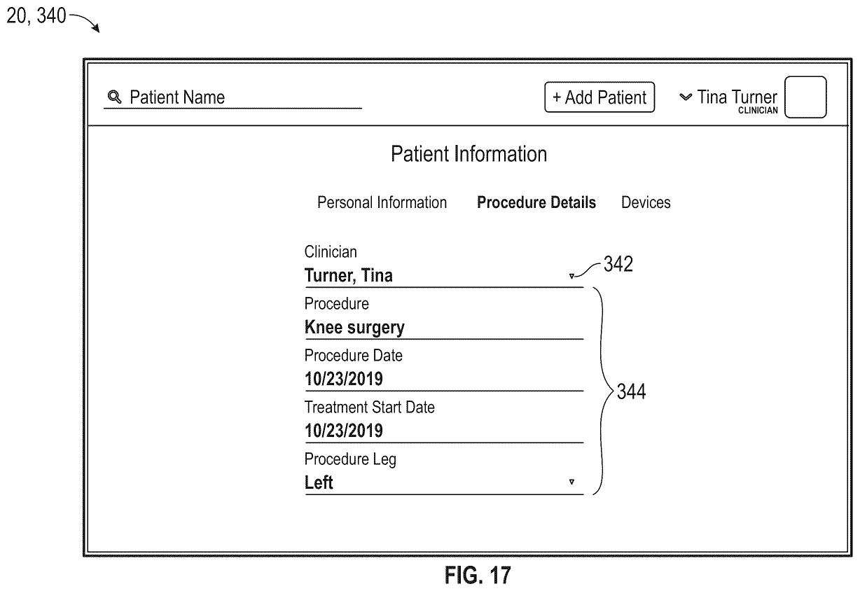

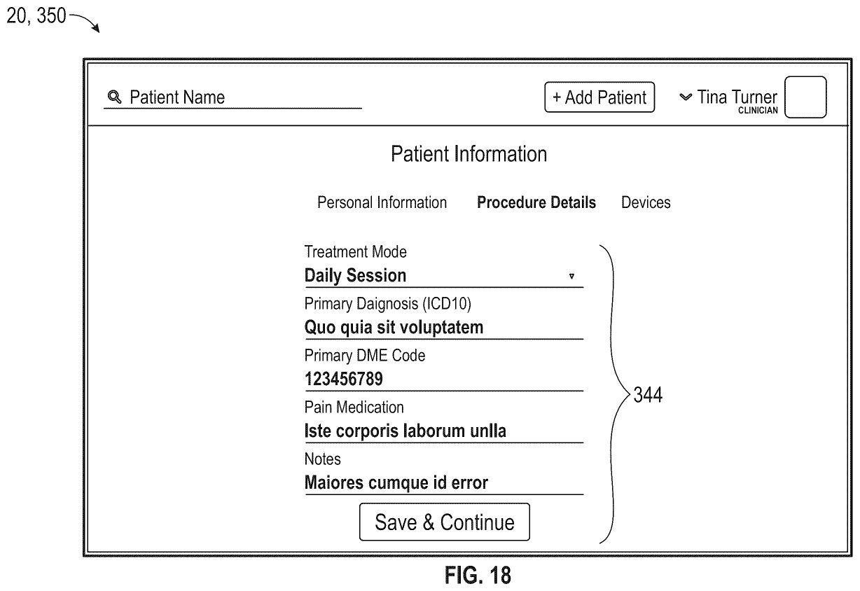

FIGS. 17-18 show example embodiments of medical data displays 340, 350 of the clinician interface 20. More specifically, the example medical data displays 340, 350 of FIGS. 17-18 are configured for modifying medical procedure information regarding a patient. The medical data displays 340, 350 may be presented using one or more screens or displays. One or more of the medical data displays 340, 350 include a clinician assignment control 342 configured to assign one or more clinicians as being associated with (e.g., responsible for) the given patient. Each of the medical data displays 340, 350 also includes a plurality of treatment information fields 344, each holding a corresponding treatment information item regarding the given patient. The treatment information items may include, for example, a type of procedure performed or to be performed upon the given patient, a procedure date, a start date for a rehabilitation treatment plan, a body part being treated, a treatment mode, a primary diagnosis or differential diagnosis (e.g., DRG, ICD10, etc.), a listing of pain medication prescribed to the given patient, etc.

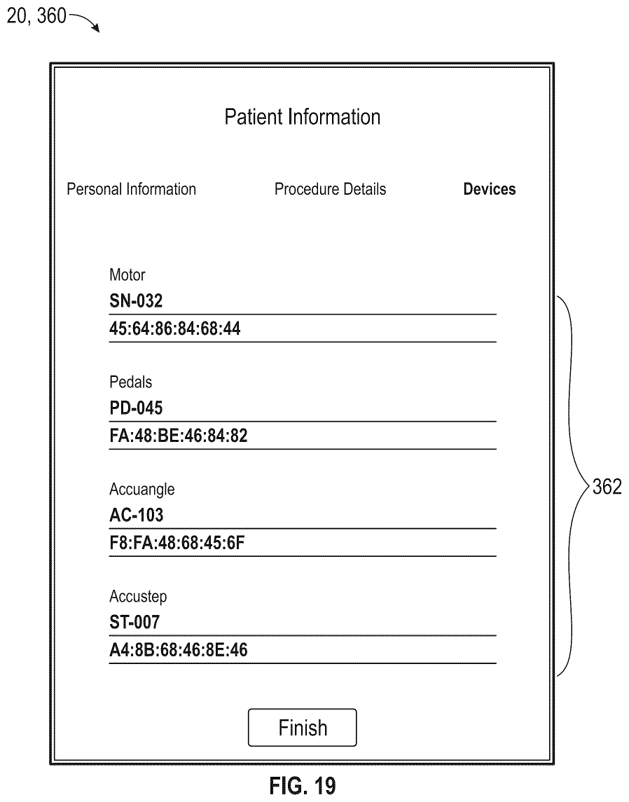

FIG. 19 shows an example embodiment of a device data display 360 of the clinician interface 20. The device data display 360 includes device data fields 362, including data regarding the treatment apparatus 70 and/or any other devices provided for use by the given patient in performing the treatment plan. The device data fields 362 may include serial numbers and other identifying information, such as a Bluetooth MAC address for various equipment such as the treatment apparatus 70 (i.e., the “Motor”), the pedals 102, the goniometer 84 (i.e., the “Accuangle”), and/or the ambulation sensor 82 (i.e., the “Accustep”).

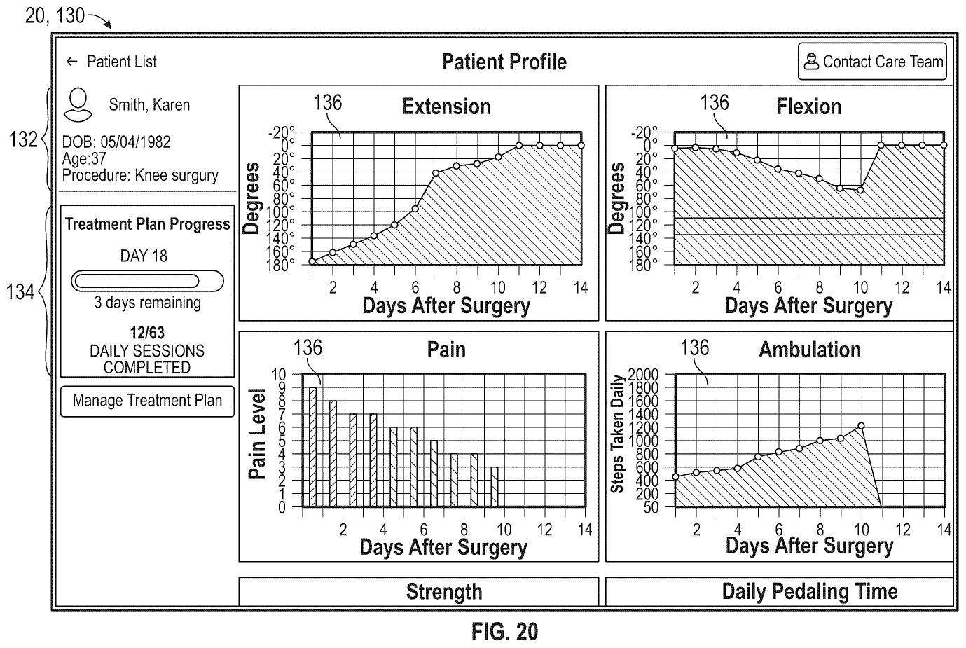

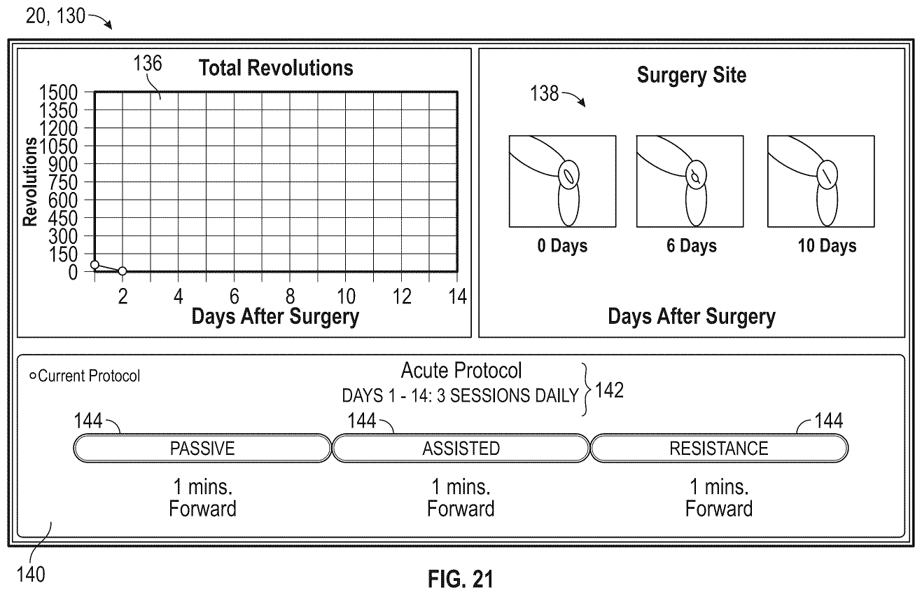

FIGS. 20-21 show an example embodiment of a patient profile display 130 of the clinician interface 20. The example patient profile display 130 includes a patient summary 132 with the patient's name, date of birth (DOB), age, a description of a procedure performed or to be performed on the patient, e.g., “Knee surgery”, and a picture of the patient, if available. The example patient profile display 130 also includes a treatment progress summary 134, showing one or more indicators of progress within a treatment regimen or plan. The example treatment progress summary 134 shown on FIG. 20 includes textual progress summaries, “DAY 18”, “3 days remaining”, “12/63 DAILY SESSIONS COMPLETED”, as well as graphical progress summaries in the form of horizontal bar graphs, which may also be called progress bars.

The example patient profile display 130 presents information regarding a treatment history of the patient. For example, the example patient profile display 130 includes a plurality of different treatment graphs 136 showing the effect of various treatment parameters over time. The treatment graphs 136 shown in the example patient profile display 130 of FIGS. 20-21 include extension (angle), flexion (angle), pain (0-10 scale), ambulation (steps/day), and total revolutions (i.e., revolutions performed on the stationary cycling machine 100). The patient profile display 130 shown on FIG. 21 also includes a pictorial history 138, showing one or more images of the surgical site for reference by a clinician or other healthcare professional in reviewing post-operative progress. The images in the pictorial history 138 may be taken by the patient and/or by a clinician or other healthcare professional. For example, the first picture may be taken by a member of the surgical staff, and subsequent pictures may be taken by the patient and/or the rehabilitation clinician. The example patient profile display 130 shown on FIG. 21 also includes a protocol summary display 140 showing a summary overview of a treatment protocol to be performed by the patient. The example protocol summary display 140 includes a protocol heading 142 with a protocol name, e.g. “Acute Protocol.” The protocol heading 142 also includes overview information regarding how and when the protocol is to be performed, e.g. “Days 1-14, 3 sessions daily.” The protocol summary display 140 also includes several protocol session icons 144, each indicating details of an activity to be performed within a protocol session, e.g., “Passive”, “Active”, or “Resistance”, together with other information regarding the protocol session, such as a direction (forward/reverse), and an amount of time that each protocol session is prescribed to be performed.

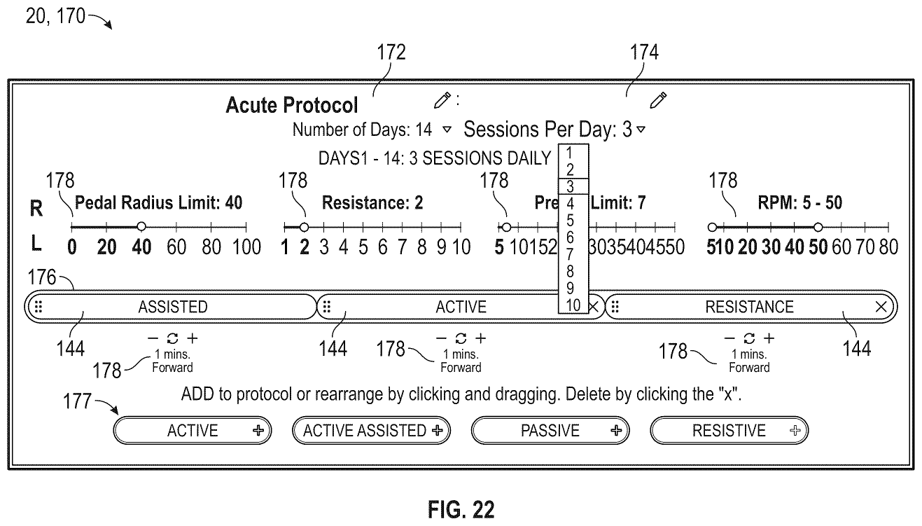

FIG. 22 shows an example embodiment of a protocol management display 170 of a clinician interface 20 for editing a treatment protocol 156. Specifically, the protocol management display 170 includes a protocol name control 172 for renaming the treatment protocol 156. The protocol management display 170 also includes a protocol timing control 174 for adjusting various timing settings of the treatment protocol 156, such as a duration for the treatment protocol 156 within the treatment plan 152, and a number of sessions to be performed per day. The example protocol timing control 174 shown on FIG. 22 includes drop-down menus for changing the various timing settings, but other controls could be used such as, for example, numeric entry fields or increase/decrease buttons. The protocol management display 170 also includes a protocol session control 176 for customizing the session periods. Specifically, the protocol session control 176 includes a graphical representation of a session, with protocol session icons 144, which may be similar or identical to the protocol session icons 144 of the protocol summary display 140. Each session period may have an associated type, such as passive, resistance, assisted, or active. Each session period may also have several parameters associated therewith.

The protocol session control 176 allows the clinician to adjust the number, the order, and the types of the session periods within a given session of the treatment protocol 156. Each session period has a type that corresponds to a category of activity to be performed upon a body part during that session period. For example, the session periods may be one of a passive period, an assisted period, an active period, or a resistance period. Each passive period is associated with a particular activity that includes moving a body part by an external force; each assisted period is associated with a particular activity that includes moving the body part by the patient with assistance of the external force; each active period is associated with a particular activity that includes the patient moving the body part without assistance of the external force; and each resistance period is associated with a particular activity that includes the patient actively moving the body part against a resistance force. For example, where the treatment apparatus 70 includes a stationary cycling machine 100, a passive period may include an actuator 78, such as a motor, that rotates the pedals 108 with the patient's feet and legs attached thereto and without any action or force being applied by the patient. An assisted period may include the patient applying force to rotate the pedals 108 with some additional help or assistance from the actuator 78. An active period may include the patient applying force to rotate the pedals 108 without any assistance from any outside force. A resistance period may include the patient exerting some force to rotate the pedals 108 in opposition to a resistance force applied by the actuator 78. In some embodiments, the actuator 78 may produce the external forces for each of the different categories of the session periods. The external forces may have different attributes, such as directions, intensities, or rates of changes, for each of the different categories of the session periods. Each session may include any number of session periods in any combination.

In some embodiments, the protocol session icons 144 may be modified using a drag-and-drop interface. Additional protocol sessions may be added to the protocol session using a session period control 177. Additionally, parameters for any or all of the session periods may be adjusted using various session parameter controls 178. For example, a duration and direction of each session period may be adjusted using the session parameter controls 178 located below an associated one of the protocol session icons 144. Various other parameters, such as resistance, target speed range (RPM), pedal radius limits, etc. may be adjusted using other session parameter controls 178. In some embodiments, the number and the type of session parameter controls 178 may change depending on the type of session period selected. For example, selecting a protocol session icon 144 for an active type of session period may cause the target speed range (RPM) session parameter control 178 to be visible and adjustable, but the target speed range (RPM) session parameter control 178 may not be visible and/or adjustable in response to selecting a protocol session icon 144 for a passive type session.

In some embodiments, the system 10 may impose limits on values that can be set using the session parameter controls 178. For example, the treatment plan 154 may include a maximum session time. In some embodiments, to satisfy a rule of the system 10 or a rule within the treatment plan 154, one or more of the values of the parameters may be automatically changed by the system 10. For example, the treatment plan 154 may require a resistance type of session period after an active type of session period, wherein the former is at least 25% as long as the active type of session to allow the patient to cool down after active exercise. The system 10 may automatically create the resistance type session period in response to the clinician creating an active type session period. The system 10 may also automatically adjust the time of the resistance type session period to satisfy the requirement of it lasting at least 25% as long as the active type of session.

In some embodiments, the treatment plan 154 may include maximum values for certain parameters until an associated condition is satisfied. For example, the pedal radius limit may be limited to 40 mm until an associated condition is satisfied. Associated conditions may include, for example, approval by an authorized person, such as an orthopedic surgeon; the elapsing of a particular time, such as 5 days after a surgical procedure; or successful completion of a post-operation checkup. Similarly, the treatment plan 154 may place limits on the types of session periods that may be performed until an associated condition is satisfied. The treatment plan 154 may be limited to only passive or assisted session periods (and not active periods or resistance periods until an associated condition is satisfied. Different associated conditions may be associated with each of the different parameters and/or with limits on the types of session periods available.

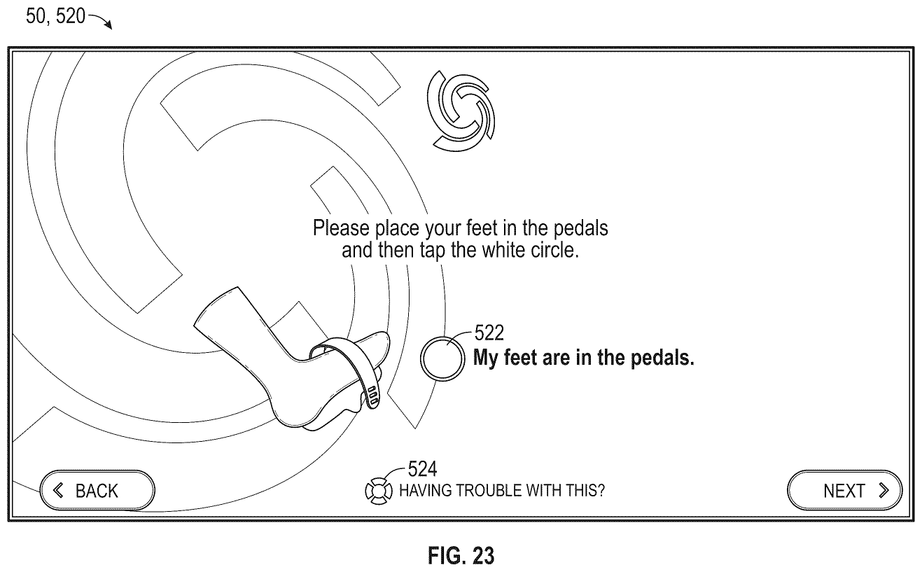

FIG. 23 shows an example embodiment of positioning confirmation screen 520 of the patient interface 50. This screen 520 is the beginning of a guided walk-through for the patient to use the treatment apparatus 70. Specifically, this screen 520 includes written instructions to guide the patient in placing their feet in the pedals 102 of a stationary cycling machine 100. In some embodiments, this screen 520 may include graphics, such as pictures or animations to help the patient perform particular actions for using the treatment apparatus 70. Screen 520 includes a position confirmation selector 522 for the patient to indicate that they are in position to use the treatment apparatus 70. Screen 520 also includes a trouble button 524 for the patient to indicate that they are having trouble getting in position to use the treatment apparatus 70.

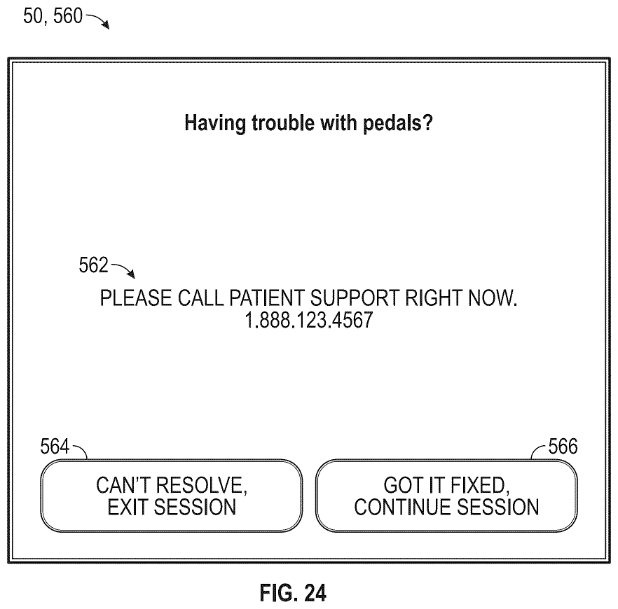

FIG. 24 shows an example embodiment of a positioning help screen 560 of the patient interface 50. This help screen 560 may be shown in response to the user selecting the trouble button 524 on the positioning confirmation screen 520. The help screen 560 may automatically be displayed if the patient fails to select the position confirmation selector 522 within a predetermined period of time. In some embodiments, an intermediate screen such as a popup asking if the patient needs more time may be displayed before the help screen 560 is shown. The help screen 560 includes assistance instructions 562 for the patient to obtain assistance for using the treatment apparatus 70. In some embodiments, the assistance instructions 562 may include a phone number. The assistance instructions 562 may also include other items, such as a link to a video conference with someone able to help the patient, and/or a link to a video or animated walk-through with detailed instructions for performing a particular action to use the treatment apparatus 70. The particular action may include, for example, placing the feet in the pedals. The help screen 560 may also include an exit button 564 that the patient can use to stop the treatment session in case they are unable to resolve their issue with using the treatment apparatus 70. Use of the exit button 564 may generate an alert to the clinician. The help screen 560 also includes a proceed button 566 that the patient can use to indicate that they have resolved their issue and are able to proceed with the treatment session.

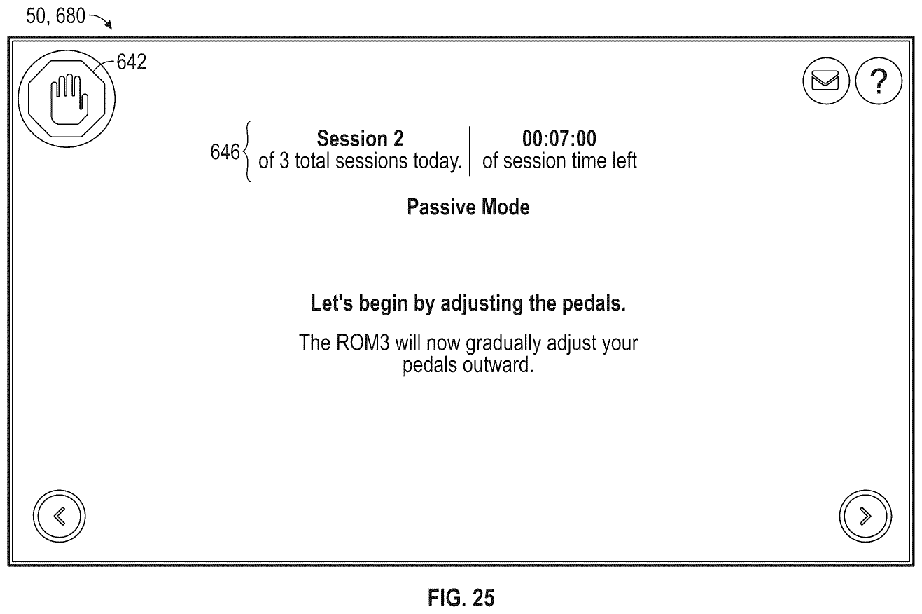

FIG. 25 shows an example embodiment of an adjustment introduction screen 680 of the patient interface 50. The adjustment introduction screen 680 includes text and/or graphics indicating various adjustments to be performed by the treatment apparatus 70. In the example shown, the adjustments include the treatment apparatus 70 that is a stationary cycling machine 100 that automatically moves the pedals 102 outwardly to a predetermined position for the session period.

In some embodiments, the patient interface 50 presents an adjustment confirmation control configured to solicit a response regarding the patient's comfort level with the position of the body part or the force exerted by the body part. The comfort level may be indicated by a binary selection (e.g., comfortable or not comfortable). In some embodiments, the comfort level may be an analog value that may be indicated numerically or with an analog input control, such as a slider or a rotary knob. In some embodiments, the comfort level may be indicated by one of several different comfort level values, such as an integer number from 1 to 5. In some embodiments, the comfort level may be indicated using controls for the patient to maintain a setting or for the patient to change the setting. More specifically, the control for the patient to change the setting may provide for the patient to change the setting in either of two or more directions. For example, the controls may allow the patient to maintain the value of a setting, to increase the value of the setting, or to decrease the value of the setting.

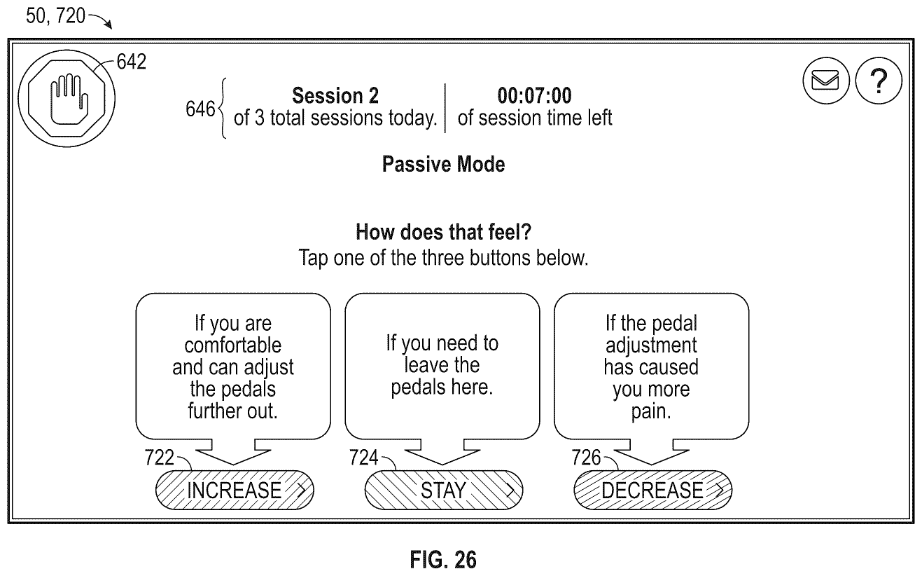

The adjustment confirmation control may take the form of an adjustment confirmation screen 720, as shown, for example, in FIG. 26. The adjustment confirmation control may take other forms, such as a popup window or a portion of a larger display screen. The patient interface 50 may present the adjustment confirmation control on a graphical user interface, such as a display screen or an overlay or virtual control within a virtual reality (VR) or augmented reality (AR) display. Additionally or alternatively, the adjustment confirmation control may include one or more physical control devices, such as buttons, knobs, sliders, etc. In some embodiments, the adjustment confirmation control may be used in conjunction with an automatic adjustment, such as an actuator 78 within the treatment apparatus 70. For example, as shown in the FIGS., an actuator 78 may change the radius of one of the pedals 102, thus changing the position of the patient's knees. The adjustment confirmation control may then solicit a response regarding the patient's comfort or discomfort with the adjusted position. In another example, the patient interface 50 may prompt the patient to apply a target pressure, such as 50 lbs. The adjustment confirmation control may then solicit a response regarding the patient's comfort or discomfort in applying the target pressure.

The phrases ‘increase control’, ‘decrease control’, and ‘stay control’, unless explicitly stated otherwise, are intended to be understood as noun phrases meaning controls that serve the functions of increasing, decreasing, or maintaining corresponding values.

The adjustment confirmation screen 720 includes text and/or graphics requesting the patient to confirm their satisfaction with the position of the treatment apparatus 70 during and/or after the automatic adjustments are made. The adjustment confirmation screen 720 includes an increase control that the patient may select to indicate a desire to increase the value of a corresponding parameter. The corresponding parameter may be a position of the treatment apparatus 70 such as the radius of the pedal 102 on the pedal arm 104. The corresponding parameter may be a setting for a force or a speed of an exercise performed as part of the regimen. For example, the corresponding parameter may be a target pressure or a target RPM speed in a given session period. The increase control may take the form of an increase button 722, such as the button shown on FIG. 26. The increase control may take other forms, such as a knob or slider control, which may be a physical device or part of a graphical user interface. The adjustment confirmation screen 720 also includes a stay control that the patient may select to indicate a desire to maintain the value of the corresponding parameter. The stay control may take the form of a stay button 724, such as the button shown on FIG. 26. The stay control may take other forms, such as a knob or slider control, which may be a physical device or part of a graphical user interface. The adjustment confirmation screen 720 also includes a decrease control that the patient may select to indicate a desire to decrease the value of the corresponding parameter. The decrease control may take the form of a decrease button 726 such as the button shown on FIG. 26. The decrease control may take other forms, such as a knob or slider control, which may be a physical device or part of a graphical user interface. For example, if the patient experiences pain or discomfort with the initial position, he or she may change the position using the decrease button 726 until the pain or discomfort is alleviated.

In some embodiments, one or more of the increase, the decrease, and/or the stay control(s) may be provided by one or more of the sensors 76, 84, 86. For example, the patient interface 50 may prompt the patient to move a body part until they start to feel discomfort, the system 10 may use one or more of the sensors 76, 84, 86 to measure the range of motion that the body part moved, and that range of motion may be used for performing the rehabilitation regimen. In another example, one or more of the sensors 76, 84, 86, such as a pressure sensor 76 and/or a goniometer 84, may measure a physical response by the patient, such as a flinch that indicates pain. A target value of the parameter may be set based upon the value of the parameter where the patient indicated pain or discomfort. That target value of the parameter may then be used for performing the rehabilitation regimen. The target value of the parameter may be set based upon a value of the parameter where the patient indicated pain or discomfort. The target parameter value may be set to X % of P, where X is a predetermined percentage, and P is the value of the parameter where the patient indicated pain or discomfort. For example, if a patient indicated pain at a pedal radius of 6.0 cm, and X is 90%, the target parameter value for the pedal position may be set to 5.4 cm, or 90% of 6.0 cm. Alternatively, the target parameter value may be set using an offset value that is added or subtracted from the value of the parameter where the patient indicated pain or discomfort. For example, if a patient indicated pain at pedal radius of 8.0 cm, and the offset value is −1.2 cm, then the target parameter value for the pedal radius may be set to 6.8 cm. Values of other parameters, such as target pressure or target speed, may be similarly adjusted.

In some embodiments, the system 10 may be configured to persuasively motivate the patient to use one or more settings for the position of the body part and/or the force exerted by the body part. For example, the patient interface 50 may show a target value or a target range for the position of the body part and/or the force exerted by the body part. In another example, the patient interface 50 may periodically encourage the patient to increase a setting for the position of the body part and/or the force exerted by the body part, particularly where that setting is below a target value or a target range. The system 10 may gradually increase a setting for the position of the body part and/or the force exerted by the body part while the patient is using the body part to perform the rehabilitation regimen. In some embodiments, the adjustment confirmation control may be presented to the patient only after the setting for the position of the body part and/or the force exerted by the body part has been actively used in performing the rehabilitation regimen for some period of time. In some embodiments, the adjustment confirmation control may not be presented to the patient, even after the setting for the position of the body part and/or the force exerted by the body part is adjusted.

In some embodiments, the patient interface 50 may present the adjustment confirmation control before the patient performs the rehabilitation regimen. Such a pre-performance adjustment allows the patient to use a confirmed or adjusted position and/or force setting while performing the rehabilitation regimen. Additionally or alternatively, the patient interface 50 may present the adjustment confirmation control during and/or after the rehabilitation regimen. For example, the adjustment confirmation screen 720 may be presented to the patient during a session or between sessions of the rehabilitation regimen. In some embodiments, the adjustment confirmation control may be presented in response to a triggering event. The triggering event may include, for example, the patient reporting pain in excess of a given value, or an inability to complete one or more activities within the treatment plan 154, or a sudden decrease in walking performed by the patient. Additionally or alternatively, the adjustment confirmation screen 720 may be presented to the patient after the patient has completed a session of the rehabilitation regimen. Such a post-session confirmation may be used to determine the patient's comfort, which may be a proxy for satisfaction with the session of the rehabilitation regimen. The post-session confirmation may be used to determine one or more settings for use in subsequent sessions. For example, an indication of “stay” or “increase” may cause a target value for position and/or pressure of the body part to be increased in subsequent sessions of the rehabilitation regimen.

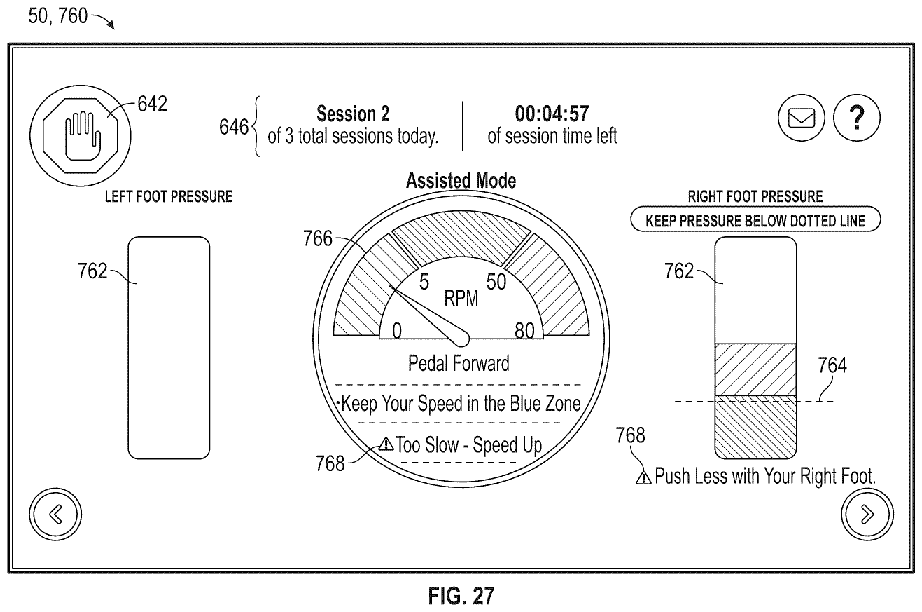

FIG. 27 shows an example embodiment of a session period action screen 760 of the patient interface 50. This screen 760 is displayed while a given session period is in progress. It includes one or more indicators showing real-time status of measurements regarding the patient's use of the treatment apparatus 70 to perform the rehabilitation regimen upon patient's body part. The measurements displayed may include, for example, a position of, and/or a force exerted by, the patient's body part. The example session period action screen 760 of FIG. 27 includes pressure indicators 762 showing an amount of pressure or force applied by each foot. The pressure indicators 762 show the pressures of the patient's feet upon the pedals 106 as measured by the pressure sensors 86. The pressure indicators 762 are shown as bar graphs, but other types of displays may be used, such as rotary gauges and/or numeric indicators. The pressure indicators 762 may also include a target pressure indicator 764 representing a target setting such as a target pressure value. The target setting may be determined by the clinician using an associated session parameter control 178 on the protocol management display 170, as shown, for example, on FIG. 22. The target setting may be set or adjusted via the adjustment confirmation control, by the patient.

In some embodiments, the clinician interface 20 may present information regarding the position of the body part and/or the force exerted by the body part. This information may include actual and/or target positions and/or forces as measured by one or more of the sensors 76, 84, 86. Additionally or alternatively, the information regarding the position of the body part and/or the force exerted by the body part may include a target value or a target range of values for either or both of the position of the body part and/or the force exerted by the body part. For example, the clinician interface 20 may provide a control for the clinician to adjust a value or a range of values as a target for a parameter such as a position, a force, or a speed used in a session or a session period or for a particular exercise within the rehabilitation regimen. Similarly, the clinician interface 20 may provide a control for the clinician to adjust minimum and/or maximum values for the parameter. For example, the patient may adjust the value of a pedal radius parameter from the preset target value up to the maximum value for that parameter, where the preset target value and the maximum value are both set by the clinician using corresponding controls on the clinician interface 20.

The session period action screen 760 also includes a speed indicator 766 showing a speed that the pedals 106 are turning, as measured by an internal sensor 76 of the stationary cycling machine 100. The speed indicator 766 is shown as a rotary gauge, but other types of displays may be used, such as a bar graph and/or a numeric indicator. The speed indicator 766 includes an optimal or desired speed range, which may be determined by the clinician using an associated session parameter control 178 on the protocol management display 170, as shown, for example, on FIG. 22. The session period action screen 760 may present prompts or messages 768 to enable the user to change the pressure and/or speed if either of those parameters is outside of a predetermined range.

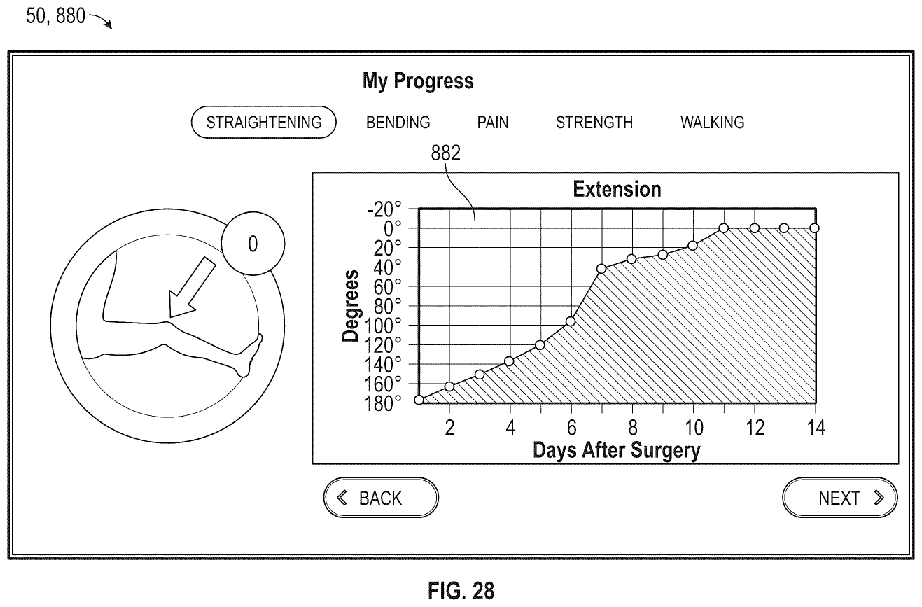

FIG. 28 shows an example progress data screen 880 of the patient interface 50. The progress data screen 880 presents a progress graph 882 for each of several different parameters related to the treatment plan 154. For example, the progress graphs 882 may include historical data for straightening and bending of the knee pain, strength (lbs. pressure), and walking (steps per day). The progress graphs 882 may show identical data or data similar to what is presented on the treatment parameter graphs 136 of the clinician interface 20.

In some embodiments, a computer, such as the server 30, is configured to automatically modify the treatment plan 154 in response to satisfaction by the patient of a predetermined condition. For example, the treatment plan 154 may be limited in speed, velocity, or pressure settings or number of sessions per day until a predetermined condition is satisfied. In another example, the treatment plan 154 may include only certain types of session periods, such as passive type exercises, until the predetermined condition is satisfied. The predetermined condition may include, for example, a successful post-operative checkup; or completion of a predetermined number of sessions or satisfying a performance benchmark within the treatment plan. Such a benchmark may include, for example, walking X number of steps in a day, or some given RPM speed or a given number of pounds of force using the treatment apparatus 70. In some embodiments, the computer is configured to increase at least one of a frequency, a duration, or an intensity of an aspect of the treatment plan 154 in response to performance or occurrence of the predetermined condition. In some embodiments, the computer is configured to decrease at least one of a frequency, a duration, or an intensity of an aspect of the treatment plan 154 in response to a performance or occurrence of the condition. The predetermined condition may include, for example, the patient reporting pain in excess of a given value, or an inability to complete one or more activities within the treatment plan 154, or a sudden decrease in walking performed by the patient.

In some embodiments, the patient interface 50 may provide a prompt to the patient in response to occurrence of the predetermined condition. For example, in a session period where the patient is expected to maintain the stationary cycling machine at a speed of between 40 and 50 RPM, the predetermined condition may include the cycling machine operating below 30 RPM for a period of 5 seconds. In that case, the patient interface 50 may provide a prompt asking the patient if they are having trouble or pain in performing the activity. The prompts may narrow down a problem. For example, if the patient is unable to perform a given activity, then a computer, such as the server 30, may automatically modify the treatment plan 154 to include activities that are easier for the patient to complete, such as only passive or only assisted session periods. Alternatively, the treatment plan 154 may be suspended until the clinician or another qualified person, such as an orthopedic surgeon, directs the system 10 to re-enable the treatment plan 154. Additionally or alternatively, the patient's responses to the prompts may generate an alert to the clinician.

In some embodiments, the system may communicate an alert message to the clinician using a communication message, such as a pager message or a text message or an email. The alert message may include pseudonymized data and/or anonymized data or use any privacy enhancing technology to prevent confidential patient data from being communicated in a way that could violate patient confidentiality requirements. Such privacy enhancing technologies may enable compliance with laws, regulations, or other rules of governance such as, but not limited to, the Health Insurance Portability and Accountability Act (HIPAA), or the General Data Protection Regulation (GDPR), wherein the patient may be deemed a “data subject”. For example, an alert message may direct the clinician that a particular type of alert exists, such as a patient reporting wound splitting, without identifying which patient made the report. The alert message may direct the clinician to check the clinician interface 20 for more specific details regarding the alert.

According to further aspects, the computer-implemented system 10 may be configured to automatically modify one or more parameters of the treatment plan based upon progress made by the patient in performing the treatment plan. For example, the server 30 may be configured to adjust one or more settings, such as frequency of sessions, a range of motion setting, and/or a pressure setting based on how the patient is progressing in the treatment plan. In some embodiments, the parameters available to be modified by the system may be adjusted within a corresponding range of values set by the clinician. For example, the clinician interface 20 may present one or more controls for the clinician to set a range of values that the system can use for each of the adjustable parameters. The system 10 may use an algorithm to add more sessions (e.g., if the patient is behind schedule). Alternatively, the system 10 may accelerate ahead to more difficult sessions if the recovery is proceeding faster than expected.

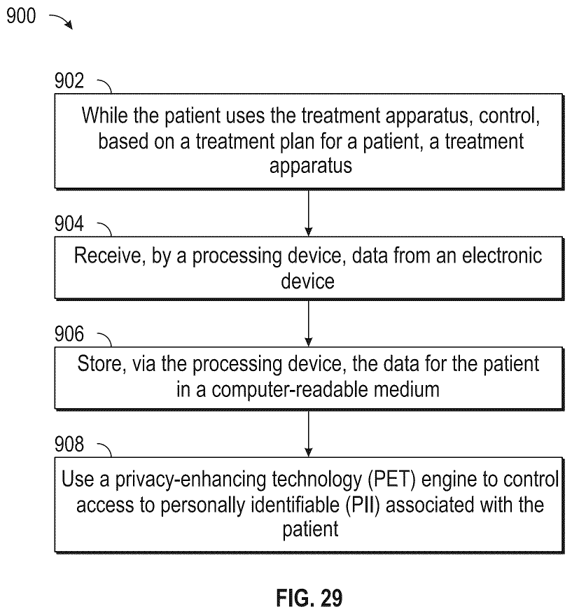

FIG. 29 shows an example flow diagram of a method 2900 for managing a treatment plan. The method 2900 is performed by processing logic that may include hardware (circuitry, dedicated logic, etc.), software (such as is run on a general-purpose computer system or a dedicated machine), or a combination of both. The method 2900 and/or each of its individual functions, routines, other methods, scripts, subroutines, or operations may be performed by one or more processors of a computing device (e.g., any component of any of the FIGs., such as interfaces, servers, treatment apparatuses, sensors, etc.). In certain implementations, the method 2900 may be performed by a single processing thread. Alternatively, the method 2900 may be performed by two or more processing threads, each thread implementing one or more individual functions or routines; or other methods, scripts, subroutines, or operations of the methods.

For simplicity of explanation, the method 2900 is depicted and described as a series of operations. However, operations in accordance with this disclosure can occur in various orders and/or concurrently, and/or with other operations not presented and described herein. For example, the operations depicted in the method 2900 may occur in combination with any other operation of any other method disclosed herein. Furthermore, not all illustrated operations may be required to implement the method 2900 in accordance with the disclosed subject matter. In addition, those skilled in the art will understand and appreciate that the method 2900 could alternatively be represented as a series of interrelated states via a state diagram, a directed graph, a deterministic finite state automaton, a non-deterministic finite state automaton, a Markov diagram, or event diagrams.

At 902, the processing device may control, based on a treatment plan for a patient, a treatment apparatus 70 while the patient uses the treatment apparatus 70. The treatment plan is for a body part, the body part may include at least one of a joint, a bone, or a muscle group. The treatment plan may include a physical rehabilitation regimen for improving strength or range of motion of the body part.

At 904, the processing device may receive, by a processing device, data from an electronic device (e.g., patient interface, computing device of an individual (patient, clinician, staff member, nurse, etc.), sensor internal or external to the treatment apparatus 70, or any some combination thereof). The data may include a measurement (e.g., pressure measurement from a sensor in a pedal of the treatment apparatus, speed of a motor operating within the treatment apparatus 70, range of motion (of a limb of the patient) received from a goniometer, etc.) pertaining to performance of a treatment plan by a patient using the treatment apparatus 70, a characteristic (e.g., a heartrate, a blood pressure, an amount of blood oxygen, a glucose level, a temperature, a perspiration rate, a pain level, etc.) pertaining to the patient, or both.

At 906, the processing device may store, via the processing device, the data for the patient in a computer-readable medium. At 908, the processing device may use a privacy-enhancing technology (PET) engine that uses privacy-enhancing technologies to control access to personally identifiable information (PII) associated with the patient. The PII may be included in the data stored in the computer-readable medium. In some embodiments, the PETs pseudonymize or anonymize the PII associated with the patient. In some embodiments, the PETs enable de-identification and re-identification of the PII associated with the patient.

In some embodiments, the processing device may maintain a set of user accounts, with each of the user accounts having an account type associated therewith. Each of the set of user accounts has a corresponding set of permissions enabling an owner of the user account to access the patient data.

In some embodiments, the processing device in a computing device of the patient or the clinician (e.g., patient interface or clinician interface) may select the treatment plan for the patient prior to controlling the treatment apparatus. In some embodiments, the processing device may select the treatment plan during a telemedicine session between a computing device of the patient (patient interface) and a computing device of a clinician (clinician interface).

### CLAUSES

Clause 1. A method comprising:
while the patient uses the treatment apparatus, controlling, based on a treatment plan for a patient, a treatment apparatus;
receiving, by a processing device, data from an electronic device, wherein the data comprises a measurement pertaining to performance of a treatment plan by a patient using a treatment apparatus, a characteristic pertaining to the patient, or both;
storing, via the processing device, the data for the patient in a computer-readable medium; and
using a privacy-enhancing technology (PET) engine to control access to personally identifiable information (PII) associated with the patient.

Clause 2. The method of clause 1, wherein the treatment plan is for a body part, the body part comprising at least one of a joint, a bone, or a muscle group.

Clause 3. The method of clause 1, wherein the treatment plan comprises a physical rehabilitation regimen for improving strength or range of motion of the body part.

Clause 4. The method of clause 1, wherein the PET engine pseudonymize or anonymize the PII associated with the patient.

Clause 5. The method of clause 1, wherein the PET engine is configured to de-identify and re-identify the PII associated with the patient.

Clause 6. The method of clause 1, further comprising:
maintaining a plurality of user accounts, with each of the user accounts having an account type associated therewith, wherein each of the plurality of user accounts has a corresponding set of permissions enabling an owner of the user account to access the patient data.

Clause 7. The method of clause 1, wherein:
the processing device is included in a computing device of a clinician or a server, and
the electronic device comprises a computing device of the patient, a sensor internal or external to the treatment apparatus, or some combination thereof.

Clause 8. The method of clause 1, further selecting, via a computing device of the patient or a clinician, the treatment plan for the patient controlling the treatment apparatus.

Clause 9. The method of clause 8, wherein the treatment plan is selected during a telemedicine session between a computing device of the patient and a computing device of a clinician.

Clause 10. A computer-implemented system for physical rehabilitation, comprising:
a clinician or patient interface comprising a patient profile display configured to present data regarding performance, by a patient, of a treatment plan for a body part, the body part comprising at least one of a joint, a bone, or a muscle group;
wherein the treatment protocol comprises a plurality of sessions for treatment of the body part of the patient;
a sensor configured to measure one of a position of the body part or a force exerted by or on the body part; and
a server configured to store patient data, the patient data including performance data regarding the performance of the treatment plan, wherein the server is controlled by a privacy-enhancing technology (PETs) engine that controls access to personally identifiable information (PII) associated with the patient.

Clause 11. The computer-implemented system of clause 10, wherein the treatment plan comprises a physical rehabilitation regimen for improving strength or range of motion of the body part.

Clause 12. The computer-implemented system of clause 10, wherein the PET engine pseudonymizes or anonymizes the PII associated with the patient.

Clause 13. The computer-implemented system of clause 10, wherein the PET engine enables de-identification and re-identification of the PII associated with the patient.

Clause 14. The computer-implemented system of clause 10, wherein the computer-implemented system is configured to maintain a plurality of user accounts, with each of the user accounts having an account type associated therewith; and
wherein each of the plurality of user accounts has a corresponding set of permissions enabling an owner of the user account to access the patient data.

Clause 15. The computer-implemented system of clause 14, wherein the account type is one of a plurality of different account types;
wherein the plurality of different account types comprises a super-administrator account type, wherein the computer-implemented system is configured to provide a user account giving the super-administrator account type at least a greater access to the patient data than any other one of the account types.

Clause 16. The computer-implemented system of clause 14, wherein the account type is one of a plurality of different account types;
wherein the plurality of different account types comprises a practice manager account type and a clinician account type;
wherein the computer-implemented system is configured to provide a user account having the clinician account type with access to modify at least some of the patient data for patients assigned to an owner of the user account having the clinician account type, wherein the computer-implemented system is configured to provide a user account having the practice manager account type, where such account type is configured to designate another one of the user accounts as the clinician account type.

Clause 17. The computer-implemented system of clause 14, wherein the account type is one of a plurality of different account types;
wherein the plurality of different account types comprises a clinician account type and a staff member account type;
wherein the computer-implemented system is configured to provide a user account having the staff member account type, wherein the staff member account type is configured to modify at least some of the patient data for patients assigned to an owner of the user account having the staff member account type, wherein the computer-implemented system is configured to enable a user account having the clinician account type to designate another one of the user accounts as the staff member account type, wherein the computer-implemented system is configured to enable the user account having the clinician account type to assign a patient to a user account having the staff member account type.

Clause 18. The computer-implemented system of clause 14, wherein the account type is one of a plurality of different account types;
wherein the plurality of different account types comprises a patient account type, wherein the computer-implemented system is configured to enable a user account having the patient account type to view the patient data associated with their own user account.

Clause 19. The computer-implemented system of clause 14, wherein the computer-implemented system is configured to use login credentials to restrict access to each of the user accounts.

Clause 20. The computer-implemented system of clause 14, wherein, using a communication channel outside of the clinician interface and in response to a notification trigger event, the server is configured to generate an alert message to a clinician;
wherein the notification trigger event is one of a reported event or a measured event, a reported event being a condition reported by the patient, and a measured event being satisfaction of a condition that includes a measurement of the patient; and wherein the alert message comprises only authorized PII of the patient.

Clause 21. A system for remote treatment, comprising:
a clinician or patient interface configured to present controls for modifying a treatment plan comprising a regimen for treatment of a body part of a patient, with the body part comprising at least one of a joint, a bone, or a muscle group;
a treatment apparatus for performing the regimen upon the body part, the treatment apparatus configured to be manipulated by the patient; and
a server configured to store patient data, the patient data including performance data regarding the performance by the patient in following the treatment plan;
wherein the patient interface and the treatment apparatus are each configured to enable operation from a patient location geographically separate from a location of the clinician interface; and
wherein the server is controlled by a PET engine that controls access to PII associated with the patient.

Clause 22. A method for displaying clinician user interface generated by a computer, comprising:
a profile display presenting information regarding a treatment history of a patient;
a protocol management display presenting a treatment plan, with the treatment plan comprising a plurality of treatment protocols;
a plan modification control configured to modify the plurality of treatment protocols of the treatment plan; and
a login interface configured to enable a person to access the clinician user interface by providing a credential associated with one of a plurality of user accounts, wherein each of the plurality of user accounts has a corresponding set of permissions controlling access to patient data on the clinician user interface.

Clause 23. The method of clause 1, further comprising:
a notification settings display configured to designate one or more trigger conditions as being enabled for a specified user account;
wherein an alert message is sent to a person having the specified user account in response to occurrence of a given trigger condition for any patients assigned to the person having the specified user account, only if the given trigger condition is designated as being enabled for the specified user account; and
wherein the alert message is sent to the person having the specified user account using a communication channel outside of the clinician user interface.

Clause 24. The method of any clause herein, further comprising:
a team management display presenting a list of user accounts associated with a practice; and
a new user add control configured to add a new user account or to associate an account type with a user account.

Clause 25. The method of any clause herein, further comprising:
a team member display configured to enable modification of characteristics of a selected user account.

Clause 26. The method of any clause herein, wherein the team member display presents an account type control input for modifying an account type associated with the selected user account.

Clause 27. The method of any clause herein, wherein the team member display presents a plurality of permission controls, wherein each of the permission controls is configured to modify an ability of the selected user account to perform an action or to view or to modify a subset of the patient data.

Clause 28. The method of any clause herein, wherein the team member display presents a plurality of permission controls, the plurality of permission controls comprising a set of account permission controls associated with a given account type;
wherein the set of account controls is configured to selectively enable an ability for the selected user account to add or modify another user account having the given account type.

Clause 29. The method of any clause herein, wherein one user account of the plurality of user accounts corresponds to a super-administrator account type, wherein the super-administrator account type has a set of permissions enabling controlling granting or revocation of access to at least a portion of PII with respect to which other entities are granted or denied the access, including the level of the access granted or denied, and to any other conditions, including time, location, identity, or some combination thereof.

Clause 30. The clinician user interface of any clause herein, wherein one user account of the plurality of user accounts corresponds to a super-administrator account type, wherein the super-administrator account type has a set of permissions enabling controlling granting or revocation of access to at least a portion of PII with respect to which other entities are granted or denied the access, including the level of the access granted or denied, and to any other conditions, including time, location, identity, or some combination thereof.

Clause 31. A computer-implemented clinician user interface for physical rehabilitation, comprising:
a profile display presenting information regarding a treatment history of a patient;
a protocol management display presenting a treatment plan, with the treatment plan comprising a plurality of treatment protocols;
a plan modification control configured to modify the plurality of treatment protocols of the treatment plan; and
a login interface configured to enable a person to access the clinician user interface by providing a credential associated with one of a plurality of user accounts, wherein each of the plurality of user accounts has a corresponding set of permissions controlling access to patient data on the clinician user interface.

Clause 32. The computer-implemented system of any clause herein, further comprising:
a notification settings display configured to designate one or more trigger conditions as being enabled for a specified user account;
wherein an alert message is sent to a person having the specified user account in response to occurrence of a given trigger condition for any patients assigned to the person having the specified user account, only if the given trigger condition is designated as being enabled for the specified user account; and
wherein the alert message is sent to the person having the specified user account using a communication channel outside of the clinician user interface.

Clause 33. The computer-implemented system of any clause herein, further comprising:
a team management display presenting a list of user accounts associated with a practice; and
a new user add control configured to add a new user account or to associate an account type with a user account.

Clause 34. The computer-implemented system of any clause herein, further comprising:
a team member display configured to enable modification of characteristics of a selected user account.

As will readily be appreciated by a person of ordinary skill of the art in light of having read the present disclosure, as used herein, actions described as being performed in real-time include actions performed in near-real-time without departing from the scope and intent of the present disclosure.

The various aspects, embodiments, implementations, or features of the described embodiments can be used separately or in any combination. The embodiments disclosed herein are modular in nature and can be used in conjunction with or coupled to other embodiments.

Consistent with the above disclosure, the examples of assemblies enumerated in the following clauses are specifically contemplated and are intended as a non-limiting set of examples.

What is claimed is:

1. A method for displaying a clinician user interface generated by a computer, the method comprising:
generating a plan modification control of the clinician user interface, wherein the plan modification control comprises:
a protocol session icon associated with a resistance value that is to be implemented at a treatment apparatus during a treatment session included in a treatment plan, and
a session parameter control associated with a duration or direction that is to be implemented at the treatment apparatus during the treatment session;
setting a credential to authorize receiving, via the clinician user interface, an adjustment to a subset of a plurality of treatment protocols of the treatment plan, wherein the credential is associated with one of a plurality of user accounts each having a corresponding set of permissions that control access to patient data;
receiving, via the clinician user interface, the adjustment to the subset of the plurality of treatment protocols of the treatment plan; and
in response to updating the treatment plan with the adjusted subset of the plurality of treatment protocols, transmitting the treatment plan to the treatment apparatus to enable a reconfiguration of the resistance setting of the treatment apparatus.

2. The method of claim 1, further comprising:
presenting a notification settings display configured to designate one or more trigger conditions as being enabled for a specified user account, wherein:
responsive to an occurrence of a given trigger condition, an alert message is sent to the specified user account for any patient assigned to the specified user account, and
the alert message is sent to the specified user account using a communication channel independent of the clinician user interface.

3. The method of claim 1, further comprising:
presenting, via a team management display of the clinician user interface, a list of user accounts associated with a practice; and
presenting a new user add control configured to add a new user account or to associate an account type with a user account.

4. The method of claim 1, further comprising:
presenting a team member display configured to enable modification of characteristics of a selected user account.

5. The method of claim 4, wherein the team member display presents an account type control input for modifying an account type associated with the selected user account.

6. The method of claim 4, wherein the team member display presents a plurality of permission controls, and each of the permission controls is configured to modify an ability of the selected user account to perform an action or to view or to modify a subset of the patient data.

7. The method of claim 4, wherein the team member display presents a plurality of permission controls, the plurality of permission controls comprising a set of account permission controls associated with a given account type, and the set of account controls is configured to selectively enable an ability for the selected user account to add or modify another user account having the given account type.

8. The method of claim 1, wherein one user account of the plurality of user accounts corresponds to a super-administrator account type, and the super-administrator account type has a set of permissions that enable granting or revoking access to at least a portion of personally identifiable information (PII) with respect to which other entities are granted or denied the access, including a level of the access granted or denied, and to any other conditions, including time, location, identity, or some combination thereof.

9. A clinician user interface generated by a computer, wherein the clinician user interface comprises:
a plan modification control comprising:
a protocol session icon associated with a resistance value that is to be implemented at a treatment apparatus during a treatment session, and
a session parameter control associated with a duration or direction that is to be implemented at the treatment apparatus during the treatment session; and
a login interface configured to:
set a credential to authorize receiving an adjustment to a subset of a plurality of treatment protocols of the treatment plan, wherein the credential is associated with one of a plurality of user accounts each having a corresponding set of permissions that control access to patient data,
wherein the plan modification control is further configured to:
receive the adjustment to the subset of the plurality of treatment protocols of the treatment plan, and
in response to updating the treatment plan with the adjusted subset of the plurality of treatment protocols, transmit the treatment plan to the treatment apparatus to enable a reconfiguration of the resistance setting of the treatment apparatus.

10. The clinician user interface of claim 9, further comprising:
a notification settings display configured to designate one or more trigger conditions as being enabled for a specified user account
wherein:
responsive to an occurrence of a given trigger condition, an alert message is sent to the specified user account for any patient assigned to the specified user account, and
using a communication channel independent of the clinician user interface, the alert message is sent to the specified user account.

11. The clinician user interface of claim 9, further comprising:
a team management display presenting a list of user accounts associated with a practice; and
a new user add control configured to add a new user account or to associate an account type with a user account.

12. The clinician user interface of claim 9, further comprising:
a team member display configured to enable modification of characteristics of a selected user account.

13. The clinician user interface of claim 12, wherein the team member display presents an account type control input for modifying an account type associated with the selected user account.

14. The clinician user interface of claim 12, wherein the team member display presents a plurality of permission controls, and each of the permission controls is configured to modify an ability of the selected user account to perform an action or to view or to modify a subset of the patient data.

15. The clinician user interface of claim 12, wherein the team member display presents a plurality of permission controls, the plurality of permission controls comprising a set of account permission controls associated with a given account type, and the set of account controls is configured to selectively enable an ability for the selected user account to add or modify another user account having the given account type.

16. The clinician user interface of claim 9, wherein one user account of the plurality of user accounts corresponds to a super-administrator account type, and the super-administrator account type has a set of permissions enabling controlling granting or revocation of access to at least a portion of personally identifiable information (PII) with respect to which other entities are granted or denied the access, including a level of the access granted or denied, and to any other conditions, including time, location, identity, or some combination thereof.

17. A computer-implemented system having a clinician user interface for physical rehabilitation, wherein the clinician user interface comprises:
a plan modification control configured to modify the plurality of treatment protocols, the plan modification control comprising:
a protocol session icon associated with a resistance value that is to be implemented at a treatment apparatus during a treatment session included in a treatment plan, and
a session parameter control associated with a duration or direction that is to be implemented at the treatment apparatus during the treatment session; and
a login interface configured to:
set a credential to authorize receiving an adjustment to a subset of a plurality of treatment protocols of the treatment plan, wherein the credential is associated with one of a plurality of user accounts each having a corresponding set of permissions that control access to patient data,
wherein the plan modification control is further configured to:
receive the adjustment to the subset of the plurality of treatment protocols of the treatment plan, and
in response to updating the treatment plan with the adjusted subset of the plurality of treatment protocols, transmit the treatment plan to the treatment apparatus to enable a reconfiguration of the resistance setting of the treatment apparatus.

18. The computer-implemented system of claim 17, wherein the clinician user interface further comprises:
a notification settings display configured to designate one or more trigger conditions as being enabled for a specified user account, wherein:
responsive to occurrence of a given trigger condition, an alert message is sent to the specified user account for any patient assigned to the specified user account, and
the alert message is sent to the specified user account using a communication channel independent of the clinician user interface.

19. The computer-implemented system of claim 17, wherein the clinician user interface further comprises:
a team management display presenting a list of user accounts associated with a practice, wherein a new user add control configured to add a new user account or to associate an account type with a user account.

20. The computer-implemented system of claim 17, wherein the clinician user interface further comprises:
a team member display configured to enable modification of characteristics of a selected user account.
